(19) United States
(12) Patent Application Publication
(10) Pub. No.: US 2026/0334888 A1
(43) Pub. Date: Oct. 29, 2026

(54) SYSTEMS AND METHODS FOR METABOLIC VALUE BASED PREDICTIONS

(71) Applicant: Welldoc, Inc., Columbia, MD (US)

(72) Inventors: Anand K. IYER, Columbia, MD (US); Abhimanyu KUMBARA, Columbia, MD (US); Junjie LUO, Columbia, MD (US); Mansur SHOMALI, Columbia, MD (US); Guodong GAO, Columbia, MD (US); Maxwell EBERT, Columbia, MD (US)

(73) Assignee: Welldoc, Inc., Columbia, MD (US)

(21) Appl. No.: 19/657,648

(22) Filed: Apr. 24, 2026

Related U.S. Application Data
(60) Provisional application No. 63/793,938, filed on Apr. 24, 2025.

(51) Int. Cl. A61B 5/00

(52) U.S. Cl. CPC A61B 5/7282; A61B 5/4866; A61B 5/7267

(57) ABSTRACT

Methods and systems are directed to detecting an anomalous event from metabolic data received from a user during a first time period, estimating event data associated with the user during the first time period, wherein the estimated event data includes food data, exercise data, medication data, energy data, or mood data associated with the user and contemporaneous with the metabolic data, wherein the estimated event data relates to unknown event data associated with the user during the first time period, classifying a negative condition based on the detected anomalous event, the metabolic data, the estimated event data, and known event data associated with the user during the first time period, and generating a mitigating action based on the negative condition.

### CROSS-REFERENCE TO RELATED APPLICATIONS

This application claims the benefit of priority to U.S. Provisional Patent Application 63/793,938 filed Apr. 24, 2025, the contents of which are incorporated herein by reference for all purposes.

### FIELD OF INVENTION

The present disclosure relates to systems and methods for analyzing metabolic data using machine learning models, and, more particularly, to using a machine learning model to estimate event data and/or predict metabolic data from sensed metabolic data, and an action machine learning model to output mitigating actions for negative health conditions.

### BACKGROUND

Chronic health conditions present ongoing challenges for both healthcare providers and users. Management of such conditions may rely on monitoring metabolic data, such as glucose levels, heart rate, heart rate variability, weight, and other physiological measurements, to inform treatment decisions and lifestyle modifications. Traditional monitoring approaches often involve episodic measurements, such as periodic readings or check-ups, which capture only isolated snapshots of a user's metabolic state at discrete points in time. These sporadic readings may not provide sufficient data density to reveal patterns, trends, or the underlying causes of metabolic fluctuations throughout a user's day.

Healthcare providers frequently must make treatment recommendations based on said episodic measurements, which may constrain their ability to identify correlations between user behaviors and metabolic responses. For instance, understanding how specific foods, physical activities, or medication timing affect glucose levels requires continuous observation rather than intermittent sampling. Without dense, continuous data streams, it becomes difficult to establish meaningful connections between a user's actions and their physiological outcomes, limiting the effectiveness of personalized treatment strategies.

Current health management systems may also place substantial burden on users to manually log behavioral data such as food intake, exercise, and medication administration. This manual data entry introduces friction into the health management process, as users may forget to log events, enter inaccurate information, or discontinue logging altogether due to the effort involved. The reliance on user compliance for data collection creates gaps in the information available for analysis and may reduce the accuracy of any insights derived from that data.

Furthermore, existing approaches may lack the capability to automatically infer user behaviors from physiological data (such as glucose data) or to translate detected patterns into actionable recommendations without substantial human intervention.

Nothing in this section should be construed as an admission of prior art or as an assertion that any prior art is material to patentability.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

In some aspects, a method may include detecting an anomalous event from metabolic data received from a user during a first time period, estimating event data associated with the user during the first time period, wherein the estimated event data includes food data, exercise data, medication data, energy data, or mood data associated with the user and contemporaneous with the metabolic data, wherein the estimated event data relates to unknown event data associated with the user during the first time period, classifying a negative condition based on the detected anomalous event, the metabolic data, the estimated event data, and known event data associated with the user during the first time period, and generating a mitigating action based on the negative condition, wherein the mitigating action mitigates the negative condition.

According to some embodiments, the estimating event data, classifying a negative condition, and generating a mitigating action may be performed by one of a same machine learning model or two or more different machine learning models. In some aspects, the mitigating action may be transmitted to a medical device, the medical device configured to perform a function upon receipt of the mitigating action.

In accordance with some aspects, the detecting the anomalous event, the estimating the event data, and the classifying the negative condition may be performed by one or more models. The one or more models may include a first model configured to detect the anomalous event, a second model configured to estimate the event data, and a third model configured to classify the negative condition. Alternatively, the one or more models may include a model configured to detect the anomalous event, estimate the event data, and classify the negative condition.

In some embodiments, the classifying may include identifying the negative condition and determining a risk-stratification of the negative condition. In some aspects, the generating the mitigating action may include triggering an automated communication via a communication channel to at least one of a contact or an emergency service.

According to some embodiments, the detecting, estimating, and classifying may be performed iteratively, and the generating the mitigating action may be performed when a negative condition is classified during two or more consecutive classifications. In some aspects, the method may further include generating a user-specific baseline profile based on the metabolic data, comparing current metabolic data to the user-specific baseline profile to identify a deviation, and detecting the anomalous event based on the deviation.

In some embodiments, estimating the event data may be performed by a generative machine learning model, and the generative machine learning model may be trained based on historical metabolic data for a cohort of users having a health condition.

In accordance with some aspects, a non-transitory computer-readable medium may have computer-executable instructions stored thereon, wherein the instructions include steps for detecting an anomalous event from metabolic data received from a user during a first time period, estimating event data associated with the user during the first time period, wherein the estimated event data includes food data, exercise data, medication data, energy data, or mood data associated with the user and contemporaneous with the metabolic data, wherein the estimated event data relates to unknown event data associated with the user during the first time period, classifying a negative condition based on the detected anomalous event, the metabolic data, the estimated event data, and known event data associated with the user during the first time period, and generating a mitigating action based on the negative condition.

According to some embodiments, a system for generating a mitigating action may include a trained machine learning model, a classifier, an action machine learning model, a memory having processor-readable instructions stored therein, and one or more processors configured to access the memory and execute the processor-readable instructions. The one or more processors may be configured to perform a method including detecting an anomalous event from metabolic data received from a user during a first time period, estimating, by the trained machine learning model, event data associated with the user during the first time period, wherein the estimated event data includes food data, exercise data, medication data, energy data, or mood data associated with the user and contemporaneous with the metabolic data, wherein the estimated event data relates to unknown event data associated with the user during the first time period, classifying, by the classifier, a negative condition based on the detected anomalous event, the metabolic data, the estimated event data, and known event data associated with the user during the first time period, and generating, by the action machine learning model, a mitigating action based on the negative condition.

In some aspects, a method may include detecting an anomalous event from metabolic data received from a user during a first time period, estimating event data associated with the user during the first time period, wherein the estimated event data includes food data, exercise data, medication data, energy data, or mood data associated with the user and contemporaneous with the metabolic data, wherein the estimated event data relates to unknown event data associated with the user during the first time period, predicting metabolic data associated with the user during a second time period based on the detected anomalous event, the received metabolic data, the estimated event data, and known event data associated with the user during the first time period, wherein the predicted metabolic data includes at least one of glucose levels measured via a continuous glucose monitoring (CGM) device, heart rate data, heart rate variability (HRV) data, ketone data, weight data, cortisol data, hormone data, body electrical data, respiratory data, blood pressure data, body temperature data, or oxygen saturation data, classifying a negative condition based on the detected anomalous event, the metabolic data, the estimated event data, known event data associated with the user during the first time period, and the predicted metabolic data, and generating a mitigating action based on the classified negative condition to mitigate occurrence of the negative condition during the second time period.

According to some embodiments, prior to classifying the negative condition, the method may include detecting that at least a first predicted metabolic data of the predicted metabolic data and a second predicted metabolic data of the predicted metabolic data indicate the negative condition.

In accordance with some aspects, a method may include receiving metabolic data from a user during a first time period, predicting metabolic data associated with the user during a second time period based on the received metabolic data, the estimated event data, and known event data associated with the user during the first time period, wherein the predicted metabolic data includes at least one of glucose levels measured via a continuous glucose monitoring (CGM) device, heart rate data, heart rate variability (HRV) data, ketone data, weight data, cortisol data, hormone data, body electrical data, respiratory data, blood pressure data, body temperature data, or oxygen saturation data, classifying a negative condition based on the metabolic data, the estimated event data, known event data associated with the user during the first time period, and the predicted metabolic data, and generating a mitigating action based on the classified negative condition to mitigate occurrence of the negative condition during the second time period.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a health management system for healthcare-related entities, according to aspects of the present disclosure.

FIG. 2 illustrates a portion of the health management system of FIG. 1, according to aspects of the present disclosure.

FIG. 3A illustrates another portion of the health management system of FIG. 1, according to aspects of the present disclosure.

FIG. 3B illustrates a block diagram of a training module configured to train machine learning models, according to aspects of the present disclosure.

FIG. 4A illustrates a block diagram of a machine learning model system for health-based predictions where one or more models are configured to estimate event data during a first time period and output a classified negative condition to one or more models (e.g., an action machine learning model, a rules based engine, etc.), according to aspects of the present disclosure.

FIG. 4B illustrates a block diagram of a machine learning model system for health-based predictions where one or more models are configured to predict metabolic data during a second time period and output a classified negative condition one or more models (e.g., an action machine learning model, a rules based engine, etc.), according to aspects of the present disclosure.

FIG. 4C illustrates a block diagram of a machine learning model system for health-based predictions where one or more models output a classified negative condition to an action machine learning model, according to aspects of the present disclosure.

FIG. 4D illustrates a block diagram of a machine learning model system for health-based predictions where one or more models output a classified negative condition to a rules-based engine, according to aspects of the present disclosure.

FIG. 4E illustrates another block diagram of a machine learning model system for health-based predictions where and one or more models output a classified negative condition to an action machine learning model or a rules-based engine, according to aspects of the present disclosure.

FIG. 4F illustrates another block diagram of a machine learning model system for health-based predictions where one or more models output a classified negative condition to an action machine learning model or a rules-based engine, according to aspects of the present disclosure.

FIG. 4G illustrates another block diagram of a machine learning model system for health-based predictions where one or more models output a classified negative condition to an action machine learning model or a rules-based engine, according to aspects of the present disclosure.

FIG. 5 illustrates inputs for the machine learning model system of FIG. 4A and FIG. 4B including metadata, glucose data, heart rate data, and other metabolic data for a user, according to aspects of the present disclosure.

FIG. 6 illustrates a training system for the machine learning model system of FIG. 4A and FIG. 4B, according to aspects of the present disclosure.

FIG. 7A illustrates a first prediction example for determining an event and predicting future glucose data, according to aspects of the present disclosure.

FIG. 7B illustrates a second prediction example for determining an event and predicting future heart rate data, according to aspects of the present disclosure.

FIG. 8 illustrates a block diagram of a first metabolic data prediction system configured to process metabolic data, according to aspects of the present disclosure.

FIG. 9 illustrates a block diagram of a second metabolic data prediction system configured to process metabolic data, according to aspects of the present disclosure.

FIG. 10 illustrates outputs for the machine learning model system of FIG. 4A and FIG. 4B including a metabolic data text report with glucose and heart rate data, text token identifiers, and numerical data, according to aspects of the present disclosure.

FIG. 11 illustrates a flowchart for generating mitigating actions using estimated event data, according to aspects of the present disclosure.

FIG. 12 illustrates a flowchart for generating mitigating actions using predicted metabolic data, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

In the discussion that follows, relative terms such as “about,” “substantially,” “approximately,” etc. are used to indicate a possible variation of ±10% in a stated numeric value. It is noted that the description set forth herein is merely illustrative in nature and is not intended to limit the examples of the subject matter, or the application and uses of such examples. Any embodiments described herein as exemplary are not to be construed as preferred or advantageous over other embodiments. Rather, as alluded to above, the term “exemplary” is used in the sense of example or “illustrative,” rather than “ideal.” The terms “comprise,” “include,” “have,” “with,” and any variations thereof are used synonymously to denote or describe a non-exclusive inclusion. As such, a process, method, article, or apparatus that uses such terms does not include only those steps, structure or elements but may include other steps, structures or elements not expressly listed or inherent to such process, method, article, or apparatus. Further, the terms “first,” “second,” and the like, herein do not denote any order, quantity, or importance, but rather are used to distinguish one element from another. Moreover, the terms “a” and “an” herein do not denote a limitation of quantity, but rather denote the presence of at least one of the referenced item. Additionally, the terms “user” and “user”, as well as “data” and “values” may be used interchangeably throughout this application.

The present disclosure relates to systems and methods for analyzing metabolic data and predicting estimated event data (e.g., past event data) and/or predicted metabolic data (e.g., future predicted metabolic values) for a user based on metabolic data. In some cases, the systems and methods may use one or more machine learning models to determine recommendations and predictions for actions (mitigating actions, user actions, etc.) based on received metabolic data.

As used herein, the term “metabolic data” may include any physiological data that reflects or influences a user's metabolic state. Metabolic data may include glucose levels measured via a continuous glucose monitoring (CGM) device, heart rate data, heart rate variability (HRV) data, ketone data, weight data, cortisol data, hormone data, body electrical data, respiratory data, blood pressure data, body temperature data, oxygen saturation data, or other physiological measurements. The metabolic data may be sensed by one or more sensors, wearable devices, or monitoring devices associated with the user. The metabolic data may be collected continuously, at regular intervals, or on-demand depending on the type of measurement and the capabilities of the sensing device. In some cases, the metabolic data is received during a first time period.

In some cases, the metabolic data may include data from multiple physiological sources that together provide a comprehensive view of the user's metabolic state. For example, the metabolic data may include glucose data from a CGM device combined with heart rate data from a wearable fitness tracker and weight data from a smart scale. The combination of multiple types of metabolic data may enable two or more models, such as a generative machine learning model and an action machine learning model, to generate more accurate predictions and more effective mitigating actions. The systems and methods described herein may process any type of metabolic data individually or in combination to estimate event data, predict future metabolic data, and generate actions to mitigate negative conditions.

In some cases, one or more models (e.g., a trained machine learning model, a generative machine learning model, a classifying machine learning model, a classifier, etc.) may output predicted metabolic data (e.g., future metabolic values) and/or estimated event data (e.g., past event data). Estimated event data may include, for example, a category and amount of food a user consumed based on inputs to the machine learning model that include metabolic data. The estimated event data may be contemporaneous with a first time period. The estimated event data may relate to unknown event data associated with the user, where unknown event data is event data that is not sensed or provided by the user. The estimated event data may fill the gap created by the unknown event data by inferring events that occurred but were not logged or otherwise recorded by the user.

A classifier (e.g., which may be part of the trained machine learning model or a separate module or model) may receive the estimated event data or predicted metabolic data from the trained machine learning model along with the metabolic data and known event data. The classifier may classify a negative condition and determine a risk-stratification based on the detected anomalous event, the estimated event data or predicted metabolic data, the metabolic data, and/or known event data associated with the user.

An action machine learning model may be trained based on historical and simulated data to output actions (e.g., mitigating actions) that meet or have a mitigation score to likely mitigate a negative condition caused by, for example, past events. The action machine learning model may receive the classification and risk-stratification from the classifying machine learning model and may process this data to generate the mitigating actions based on at least the classification and risk-stratification.

According to aspects of the present disclosure, a machine learning model may be trained based on a training data set that includes metabolic data for a user. The training data set may further include event data such as a user's medication type, dosage, frequency, timing, and compliance, a user's food intake type, amount, timing, or frequency, a user's exercise type, frequency, and timing, a user's metabolic or biographic trends, mood data, energy data, or combinations thereof. The training data may further include historical or simulated subsequent metabolic data and subsequent event data. The machine learning model may be trained to predict subsequent metabolic data by adjusting weights, biases, synapses, layers, nodes, or other parameters based on past metabolic data and event data from the training data set.

With respect to the actions output via, for example, the action machine learning model, the actions may include recommended food, exercise, and medicine information, automatically administering of a dosage of medication and an output time, sending an alert to a user to consume a type of food, administering a medication, facilitating a food order, activating and configuring a web or mobile application, or recommending performance of a physical activity at a given time at a given amount or dosage. In some cases, the action may include triggering a medical device such as an insulin pump to output a given dosage of insulin at the output time.

As an example, a user's CGM data over a 24-hour time period (e.g., a first time period) may be detected by a CGM device in 5 minute increments over the 24-hour time period. The CGM data may be provided as an input to a machine learning model. The machine learning model may detect events from the CGM data, such as glucose spikes, overeating events, or skipped medication events, based on characteristic patterns in the CGM data. The machine learning model may output estimated event data including a category of food such as high carbohydrate food, timing, and an amount of the category of food, based on the past CGM data. The estimated event data may be contemporaneous with the detected events from the CGM data. A classifier (e.g., classifying machine learning model or other module) may classify a negative condition such as hyperglycemia, hypoglycemia, increase in heart rate, or decrease in heart rate, and may determine a risk-stratification of the negative condition as low risk, moderate risk, high risk, or critical risk. Based on the category, timing, and amount of the category of food, and the classification and risk-stratification, the machine learning model or a different machine learning model may be used to output an action to mitigate the occurrence of the negative condition.

As another example, a user's heart rate data over a 24-hour time period may be detected by a wearable device. The heart rate data may be provided as an input to the machine learning model. The machine learning model may detect events from the heart rate data, such as elevated heart rate episodes or abnormal heart rate variability patterns. The machine learning model may output estimated event data including a type of physical activity, timing, and duration of the physical activity, based on the past heart rate data. A classifier (e.g., a classifying machine learning model or other module) may classify a negative condition such as lightheadedness and may determine a risk-stratification of the negative condition. Based on the estimated event data and the classification and risk-stratification, the machine learning model or a different machine learning model may be used to output an action to mitigate the occurrence of the negative condition. As yet another example, a user's weight data over a 7-day time period may be detected by a smart scale. The weight data may be provided as an input to the machine learning model. The machine learning model may output estimated event data including dietary patterns or water retention events based on the past weight data. Based on the estimated event data, the machine learning model or a different machine learning model may be used to output an action to mitigate the occurrence of a negative condition such as unintended weight gain or unintended weight loss.

As another example, the systems and methods may be applied to heart failure management. A user's heart data, such as electrocardiogram (ECG) data, may be received and monitored over a time period. The machine learning model may detect events from the ECG data, such as a trigger event in a downward portion of the heartbeat that may indicate atrial fibrillation (Afib). A generative machine learning model, such as a GPT-based model, may be used to fill in context data to determine why the detected event occurred. For instance, the generative machine learning model may infer that the user consumed a high sodium meal, which caused the anomaly in the ECG data. The generative machine learning model may output estimated event data including dietary events such as high sodium intake based on the detected trigger event and patterns in the ECG data. A classifier (e.g., a classifying machine learning model or other module) may classify a negative condition such as atrial fibrillation and may determine a risk-stratification of the negative condition based on the detected events and estimated event data. Based on the estimated event data and the classification and risk-stratification, the machine learning model or a different machine learning model may output an action to mitigate the occurrence of the negative condition, including recommending sodium restriction, adjusting medication dosage, or alerting a healthcare provider.

In some cases, the systems and methods disclosed herein may bridge metabolic data and mental health data. For example, research has shown that glycemic variability is linked to mood, anxiety, and depression symptoms. The systems and methods may correlate glucose variability with mood states, energy levels, and other mental health indicators to provide real-time feedback and coaching. The estimated event data may include mood data comprising physiological data indicative of an emotional state of the user, biometric data correlated with mood of the user, heart rate variability data, skin conductance data, speech data, facial expression data, user-reported mood data, or combinations thereof. The estimated event data may also include energy data comprising physiological data indicative of an energy state of the user, biometric data associated with fatigue of the user including, for example, sleep duration, sleep quality metrics, activity levels, step counts, sedentary time periods, or physiological indicators of tiredness such as reduced heart rate variability or altered movement patterns, metabolic expenditure data, physiological parameters comprising heart rate, heart rate variability, respiration rate, skin conductance, blood oxygen level, body temperature, or movement intensity of the user, user-reported energy level data, or combinations thereof.

The systems and methods may employ a cascading architecture of one or more models within two or more phases. A first phase may comprise one or more models configured to detect events from metabolic data received during a first time period, estimate events of interest during the first time period and/or predict future metabolic data for a second time period subsequent to the first time period, and identify a negative condition. The detected events may include glucose spikes, overeating, skipped medications, elevated heart rate episodes, abnormal heart rate variability patterns, or poor night sleep. The negative condition may be identified by a classifier that also determines a risk-stratification of the negative condition. The negative condition may be associated with the detected event and known and/or estimated events of interest during the first time period. Additionally or alternatively, the negative condition may be associated with the detected event, known and/or estimated events of interest during the first time period, and predicted metabolic values of the second time period. A second phase may comprise one or more models configured to generate guidance or actions, including coaching, recommendations, medical interventions, and the like, based on at least the classification and risk-stratification of the negative condition. The guidance or actions may be further based on the detected event, the known and/or estimated events of interest during the first time period and/or the predicted metabolic values from the second time period.

The classifier in the first phase may be implemented using various approaches depending on the risk level of the application and the type of negative condition being classified. In some cases, the classifier may be implemented as a rules-based engine that applies threshold-based logic to determine whether metabolic data indicates a negative condition. For high-risk applications, such as glucose management or blood pressure management, a rules-based engine may apply threshold-based logic to classify negative conditions based on predetermined clinical thresholds. For example, the rules-based engine may classify a negative condition when glucose values exceed a threshold such as approximately 250 mg/dL or when blood pressure values exceed clinically significant thresholds. For lower-risk applications, such as weight management, a machine learning model may be used to classify negative conditions based on patterns in the metabolic data. In some cases, the classifier may be implemented as a large language model (LLM) that serves as a judge to evaluate whether metabolic data indicates a negative condition. This LLM-as-judge approach may provide flexibility in adapting to changing clinical guidelines or regulatory thresholds without requiring manual updates to a rules-based engine. For example, if clinical guidelines or regulatory thresholds change, the LLM may adapt to the updated thresholds based on updated prompts or context information without requiring modifications to the underlying classification logic.

In some cases, when the one or more models (e.g., a trained machine learning model, a generative machine learning model, etc.)output predicted metabolic data for future time periods (e.g., a second time period), the system may implement successive negative detection to reduce false positive alerts. When predicting future metabolic data values at regular intervals, such as approximately every 5 minutes, the system may avoid alerting the user if a classified negative condition is expected to resolve shortly or may be a false-positive. The system may be configured to detect successive negative predictions before passing the classification to the action machine learning model or to a rules-based engine. For example, when a first predicted metabolic data value indicates a negative condition, the system may record this as a first successive negative detection (N=1). When a second predicted metabolic data value at a subsequent interval also indicates a negative condition, the system may record this as a second successive negative detection (N=2). The system may continue to track successive negative detections until a threshold number of successive negatives is reached, such as three successive negative detections (N=3), before classifying the negative condition and passing the classification to the action machine learning model. This successive negative detection approach may reduce unnecessary alerts when predicted negative conditions are transient and expected to resolve without intervention.

The systems and methods disclosed herein provide technical improvements to health management computing systems by enabling automated inference of user behaviors from physiological data without requiring manual data entry. Conventional health management systems require users to manually log behavioral data such as food intake, exercise, and medication administration, which introduces friction, reduces compliance, and creates gaps in the information available for analysis. The one or more models (e.g., a trained machine learning model, a generative machine learning model, etc.) described herein addresses this technical problem by processing continuous metabolic data streams, such as glucose data from a CGM device, heart rate data from a wearable device, or weight data from a smart scale, and automatically generating estimated event data that identifies the type, timing, and characteristics of events that likely caused observed metabolic changes. The type of event may include, for example, food consumption events such as meals or snacks, medication administration events such as insulin injections or oral medication intake, exercise events such as walking, running, or resistance training, sleep events such as sleep onset or awakening, and stress events such as physiological or psychological stressors. The timing of the event may include, for example, a timestamp indicating when the event occurred, a time range during which the event took place, a duration of the event, or a temporal relationship to other events such as time elapsed since a prior meal or medication dose. The characteristics of the event may include, for example, for food consumption events, the macronutrient composition such as high carbohydrate, medium carbohydrate, or low carbohydrate content, the glycemic index or glycemic load of the food, the portion size or caloric content, and the food category such as breakfast, lunch, dinner, or snack; for medication events, the medication type such as rapid-acting insulin, long-acting insulin, or oral hypoglycemic agent, the dosage amount, and the route of administration; for exercise events, the exercise type such as aerobic or anaerobic, the intensity level such as light, moderate, or vigorous, and the duration of the activity; and for stress events, the stress intensity level and the physiological indicators associated with the stress response. This automated event inference reduces the burden on users, improves data completeness, and enables the health management system to function effectively even when users do not manually log events.

The one or more models (e.g., a trained machine learning model, a generative machine learning model, etc.) may also output predicted metabolic data that forecasts future physiological states based on current and historical metabolic data patterns and current and historical event data. The predicted metabolic data enables the health management system to anticipate negative conditions before they occur, allowing for proactive interventions rather than reactive responses. For example, the one or more models (e.g., a trained machine learning model, a generative machine learning model, etc.) may predict that a user's glucose levels will exceed a hyperglycemic threshold within the next two hours based on current glucose trajectory and detected meal patterns, enabling the action machine learning model to recommend a preemptive intervention such as physical activity or medication adjustment. Similarly, the one or more models (e.g., a trained machine learning model, a generative machine learning model, etc.) may predict elevated heart rate or abnormal heart rate variability patterns that indicate impending fatigue or stress, allowing the system to recommend rest or stress management techniques before the negative condition manifests. This predictive capability transforms the health management system from a monitoring tool into an anticipatory system that can prevent adverse health events rather than merely detecting and responding to them after they occur.

The cascading architecture of the one or more models (e.g., a trained machine learning model, a generative machine learning model, etc.) and the action machine learning model provides a technical solution for translating detected patterns in metabolic data into actionable recommendations without substantial human intervention. The one or more models (e.g., a trained machine learning model, a generative machine learning model, etc.) processes time-series metabolic data using transformer-based architectures or other neural network architectures to identify characteristic signatures associated with different types of events, such as postprandial glucose responses indicative of food consumption or elevated heart rate patterns indicative of physical activity. The action machine learning model then receives the classification and risk-stratification of the negative condition from the classifying machine learning model and generates mitigating actions tailored to address the classified negative conditions. This cascading approach enables the health management system to provide proactive interventions based on inferred past events and predicted future metabolic states, rather than merely reacting to episodic measurements that capture only isolated snapshots of the user's metabolic state.

The technical improvements provided by the disclosed systems and methods result in a health management system that can process dense, continuous data streams from multiple physiological sources, establish meaningful connections between user actions and physiological outcomes, and generate personalized mitigating actions to prevent or address negative health conditions. The machine learning models are trained on historical metabolic data and event data to learn the relationships between metabolic patterns and underlying events, enabling the models to generalize across different users and disease types. The system formed by the sensing devices, the one or more models (e.g., a trained machine learning model, a generative machine learning model, a classifying machine learning model, a classifier, etc.), and the action machine learning model provides an improved technology for mitigating negative conditions based on metabolic data without requiring material human intervention, thereby addressing the limitations of conventional health management approaches that rely on episodic measurements and manual data entry.

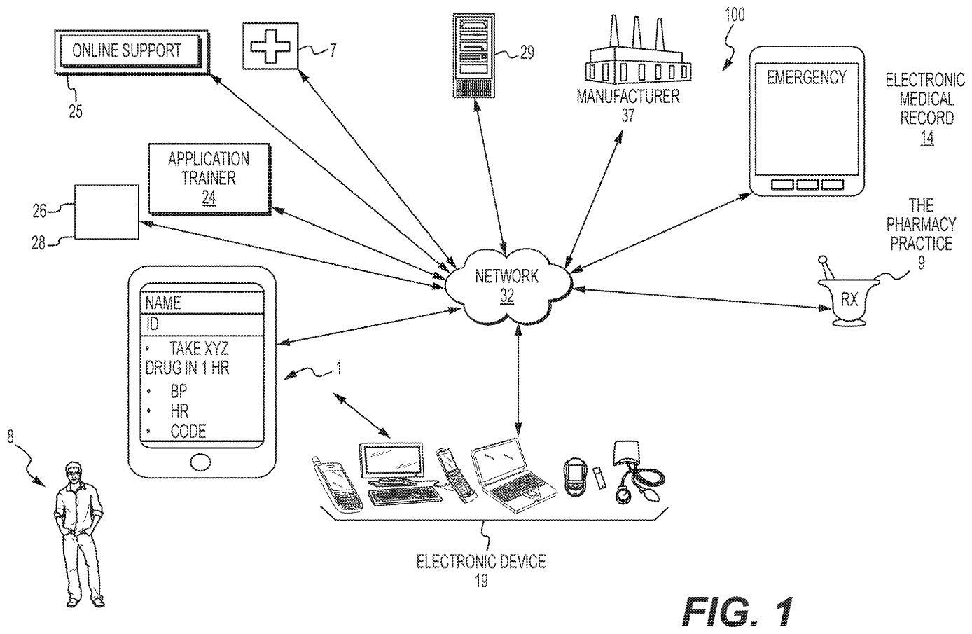

Referring to FIG. 1, a health management system 100 may be configured to facilitate communication and data exchange between various healthcare-related entities. The health management system 100 may include several components connected through a network 32, which may serve as a central communication hub for the health management system 100.

A user (e.g., patient) 8 may interact with the health management system 100 through one or more electronic devices 19. The electronic devices 19 may include mobile phones, tablets, laptops, desktop computers, smartwatches, wearable devices, and medical monitoring devices. The electronic devices 19 may be configured to access and communicate with a mobile health application 1. The mobile health application 1 may display information including user identification, medication reminders, blood pressure readings, heart rate data, and access codes. In some cases, the mobile health application 1 may be implemented as a cloud-based service accessible through the electronic devices 19.

The network 32 may connect the electronic devices 19 to multiple external entities and services. A healthcare provider 7 may connect to the network 32 to provide medical services and oversight. The healthcare provider 7 may access user data and treatment plans through the network 32. A support system 25 may connect to the network 32 to provide online support functionality for the user 8. An application trainer 24 may connect to the network 32 to facilitate user training on the mobile health application 1.

The mobile health application 1 may be associated with programming 28 and machine learning programming algorithms 26. The programming 28 and the machine learning programming algorithms 26 may enable intelligent processing and analysis of health data received through the health management system 100. The machine learning programming algorithms 26 may include algorithms for training and executing the generative machine learning model and the action machine learning model described herein.

As further shown in FIG. 1, servers 29 may connect to the network 32 to provide data storage and processing capabilities for the health management system 100. The servers 29 may store and process metabolic data, event data, and predicted data generated by the machine learning models. A manufacturer 37 may connect to the network 32 to provide information regarding medical products and therapies. The manufacturer 37 may provide data related to medications, medical devices, and treatment protocols.

A pharmacy 9 may connect to the network 32 to handle prescription dispensing and medication refills. The pharmacy 9 may receive automated orders for medications based on mitigating actions output by the action machine learning model. An electronic medical record 14 may store user health information and connect to the network 32. The electronic medical record 14 may be updated based on actions, predicted event data, and metabolic data processed by the health management system 100 (e.g., programming 28 and machine learning programming algorithms 26).

The health management system 100 may employ a star topology configuration, with the network 32 serving as the central hub through which all components communicate. Data may flow between the user 8 and the various healthcare entities through the electronic devices 19 and the network 32, enabling real-time health monitoring, medication management, and communication with the healthcare provider 7. The mobile health application 1 may receive and display health-related data, provide reminders and recommendations, and facilitate interaction between the user 8 and the healthcare ecosystem.

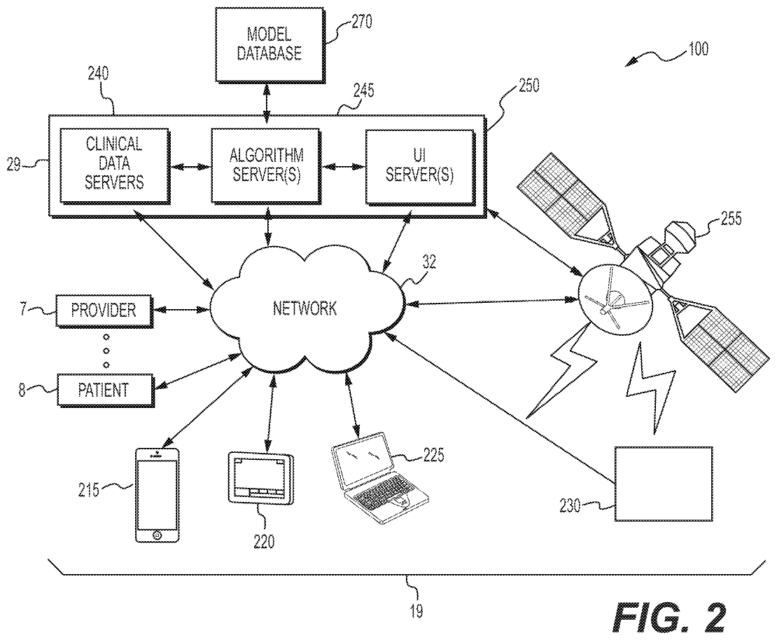

Referring to FIG. 2, a portion of the health management system 100 may include additional aspects of the system architecture. The health management system 100 may include a decision model database 270 that is accessible via the network 32. The decision model database 270 may store decision models that are retrieved for display and processing by one or more electronic devices 19. The decision models stored in the decision model database 270 may include models for determining actions, predicting metabolic data, and correlating event data with metabolic data, such as the one or more models (e.g., a trained machine learning model, a generative machine learning model, etc.) and the action machine learning model described herein.

The electronic devices 19 may include a mobile device 215, a tablet device 220, a computer 225, and a kiosk 230. The mobile device 215 may be a smartphone or other handheld computing device configured to execute the mobile health application 1. The tablet device 220 may be a portable computing device with a larger display than the mobile device 215. The computer 225 may be a desktop or laptop computing device. The kiosk 230 may be located at a pharmacy, clinic, or hospital having medical and prescription information. Each of the electronic devices 19 may be connected to the network 32 for sending and receiving data related to metabolic data, event data, and actions.

With continued reference to FIG. 2, the mobile device 215, the tablet device 220, and the computer 225 may each be equipped with a global positioning system receiver for obtaining and reporting location information via the network 32 to and from the servers 29 and one or more GPS satellites 255. The GPS satellites 255 may be configured to send and receive information between the servers 29 and the electronic devices 19. Location information obtained from the GPS satellites 255 may be used by the health management system 100 to provide location-based services, such as identifying nearby pharmacies for medication orders or determining environmental factors that may influence metabolic data.

The servers 29 may include a clinical data server 240, an algorithm server 245, and a user interface server 250. Each of the electronic devices 19, including the mobile device 215, the tablet device 220, the computer 225, and the kiosk 230, may be configured to send and receive data to and from the servers 29 over the network 32. Each server may access the decision model database 270 to retrieve decision models for processing metabolic data and generating actions.

The clinical data server 240 may have a processor configured to retrieve clinical data from a provider database or a user electronic medical record. The clinical data server 240 may store and process metabolic data received from CGM devices, heart rate monitors, smart scales, and other monitoring devices. The algorithm server 245 may have a database that includes various algorithms and a processor configured to process the clinical data. The algorithm server 245 may execute the one or more models (e.g., a trained machine learning model, a generative machine learning model, a classifying machine learning model, a classifier, etc.) and the action machine learning model to generate estimated event data, predicted metabolic data, and actions. The user interface server 250 may be configured to receive and process input from the user 8, such as clinical decision preferences, mood data, and energy data.

The healthcare provider 7 may access the health management system 100 through the network 32 to interact with user data and treatment plans. The user 8 may access the health management system 100 through any of the electronic devices 19 to receive health-related information and provide feedback. Communication between the healthcare provider 7 and the user 8 may be facilitated through the network 32 and the servers 29.

In some cases, the health management system 100 may use a conversational front end to the mobile health application 1 where the user 8 can speak to the mobile health application 1 instead of using typical graphical user interface inputs. The conversational front end may allow the user 8 to provide voice input regarding glucose readings, food intake, medication administration, mood states, and energy levels. The conversational front end may process natural language input and provide spoken responses regarding actions and health recommendations.

The health management system 100 may integrate with rewards and badges where the user 8 receives points for tracking mood and energy levels. The rewards and badges may encourage the user 8 to log mood and energy data, which may improve the accuracy of the one or more models (e.g., a trained machine learning model, a generative machine learning model, etc.) in generating estimated event data. The health management system 100 may provide a daily check-in notification if a threshold number of metabolic data excursions occur without logging by the user 8. The daily check-in notification may prompt the user 8 to log mood and energy levels and to take action to prevent metabolic data excursions.

Mood and energy trackers may be integrated into a metabolic data graph display presented through the mobile health application 1. The metabolic data graph on a home screen of the mobile health application 1 may include correlation messaging between mood and energy levels and metabolic data such as glucose data or heart rate data. The correlation messaging may help the user 8 understand the relationship between metabolic data and mood or energy states.

The health management system 100 may use engagement data including user medication intake, diet, physical activity, laboratory results, and education activity as MEDAL activities. MEDAL may refer to medication, exercise, diet, activity, and lifestyle factors that influence metabolic data. The health management system 100 may determine an engagement state as either a high engagement state or a low engagement state based on the engagement data. The engagement state may be used to adjust the frequency and type of notifications and actions provided to the user 8 from the mobile health application 1.

The health management system 100 may determine metabolic data engagement based on a threshold number of metabolic data readings collected during, for example, a first time period (1 minute, 1 day, 1 month, etc.). For example, if a CGM device recorded approximately 70 percent or more of possible CGM readings, the metabolic data engagement state may be classified as a high engagement state. Similarly, if a heart rate monitor recorded approximately 70 percent or more of possible heart rate readings, the metabolic data engagement state may be classified as a high engagement state. The metabolic data engagement state may influence the confidence level of predictions generated by the one or more models (e.g., a trained machine learning model, a generative machine learning model, a classifying machine learning model, a classifier, etc.) and the action machine learning model. A high engagement state may result in more accurate predictions due to the availability of more complete metabolic data.

The health management system 100 may adjust the confidence level of the estimated event data based on the engagement state by calculating a data completeness ratio from the engagement state. The data completeness ratio may represent the ratio of actual metabolic data readings collected to the total number of possible metabolic data readings during the first time period. For example, if a CGM device is configured to record one reading approximately every 5 minutes, the total number of possible readings during a 24-hour period may be approximately 288 readings. If the CGM device recorded 202 readings during the 24-hour period, the data completeness ratio may be calculated as approximately 0.70 (202 divided by 288). The health management system 100 may apply a scaling factor or weighting factor to the confidence level based on the data completeness ratio. When the engagement state is classified as a high engagement state, such as when the data completeness ratio is approximately 0.70 or greater, the confidence level may be maintained at a baseline level or may be increased. When the engagement state is classified as a low engagement state, such as when the data completeness ratio is below approximately 0.70, the confidence level may be discounted or reduced proportionally to the data completeness ratio. In some cases, the confidence level may decrease linearly based on the data completeness ratio, such that a data completeness ratio of 0.50 results in a confidence level that is 50 percent of the baseline confidence level. In some cases, the confidence level may decrease non-linearly based on the data completeness ratio, such that confidence levels decrease more rapidly as the data completeness ratio falls below certain thresholds. The adjusted confidence level may influence downstream processing by the one or more models (e.g., a trained machine learning model, a generative machine learning model, a classifying machine learning model, a classifier, etc.) and the action machine learning model, such as determining whether mitigating actions are triggered, how mitigating actions are prioritized, or whether additional user confirmation is requested before performing a mitigating action. For example, when the adjusted confidence level falls below a confidence threshold, the health management system 100 may request confirmation from the user 8 before performing a mitigating action, or may reduce the priority of the mitigating action relative to other actions with higher confidence levels.

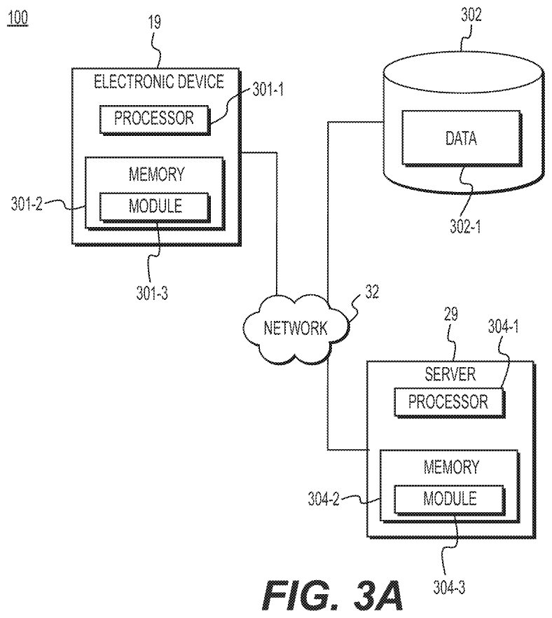

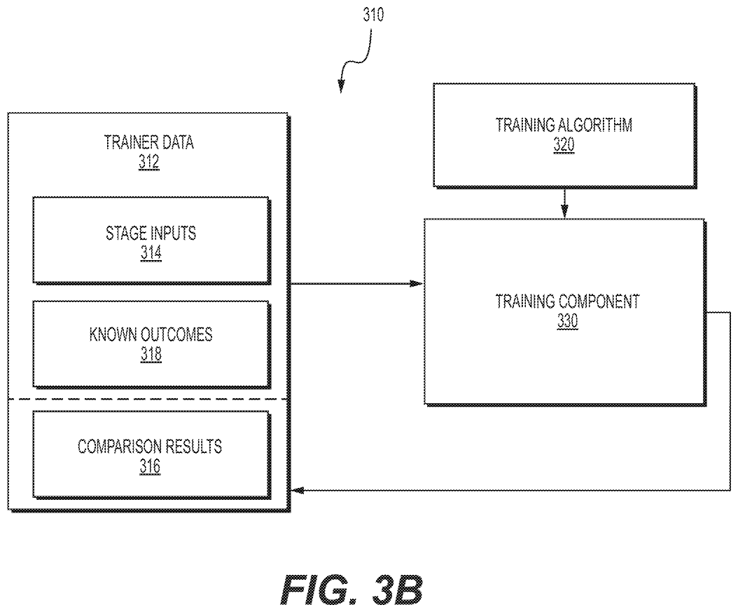

Referring to FIG. 3A, a portion of the health management system 100 may include additional details of the electronic device 19 and a server 29. The health management system 100 may be configured for data communication and processing between the electronic device 19 and the server 29 via the network 32.

The electronic device 19 may include a processor 301-1 and a memory 301-2. The memory 301-2 may store a module 301-3, which may be executed by the processor 301-1. The processor 301-1 may be a central processing unit, a microprocessor, a general purpose processor, an application specific processor, or any device that executes instructions. The memory 301-2 may be implemented using any computer-readable storage medium, such as hard drives, CDs, DVDs, flash memory, RAM, ROM, or similar storage media. The module 301-3 may include software instructions for executing functions related to metabolic data processing, event detection, and action generation.

With continued reference to FIG. 3A, the server 29 may include a processor 304-1 and a memory 304-2. The memory 304-2 may store a module 304-3, which may be executed by the processor 304-1. The server 29 may be connected to the network 32, enabling communication with the electronic device 19. The processor 304-1 may execute the one or more models (e.g., a trained machine learning model, a generative machine learning model, a classifying machine learning model, a classifier, etc.) and the action machine learning model to process metabolic data and generate estimated event data, predicted metabolic data, and actions. The module 304-3 may include software instructions for training and executing machine learning models, processing metabolic data, and generating actions based on estimated event data or predicted metabolic data.

The module 301-3 may further include instructions for rendering graphical user interfaces on the electronic device 19, processing user inputs such as mood data and energy data, and transmitting metabolic data to the server 29 for analysis by one or more models (e.g., a trained machine learning model, a generative machine learning model, a classifying machine learning model, a classifier, etc.) and an action machine learning model. The module 304-3 may further include instructions for receiving metabolic data from the electronic device 19, coordinating with the clinical data server 240 and the algorithm server 245 to retrieve clinical data and execute algorithms, and transmitting mitigating actions to the electronic device 19 or to medical devices such as insulin pumps for automated medication delivery.

The health management system 100 may also include a database 302 that stores data 302-1. The database 302 may be implemented using any database technology, such as relational database technology or object-oriented database technology. The data 302-1 may include a knowledge base for making inferences, statistical models, and user information. The knowledge base may include rules and relationships for correlating metabolic data with event data, mood data, and energy data. The statistical models may include trained parameters for the one or more models (e.g., a trained machine learning model, a generative machine learning model, a classifying machine learning model, a classifier, etc.) and the action machine learning model. The user information may include user demographics, medical history, medication schedules, and historical metabolic data. The data 302-1, or portions thereof, may be alternatively or simultaneously stored in the server 29 or the electronic device 19.

The network 32 may facilitate communication between the electronic device 19, the server 29, and the database 302. The network 32 may include wireless or wired links, such as mobile telephone networks, Wi-Fi, LANs, WANs, Bluetooth, near-field communication, or other suitable forms of network communication. The electronic device 19 may communicate with the server 29 through the network 32 to send and receive data, including clinical information and user-related data for health management purposes. The network 32 may also facilitate communication with, for example, the GPS satellites 255 for obtaining location information.

In some cases, the health management system 100 may update at least one of an electronic medical record or a user portal based on the action (e.g., mitigating action) output by the action machine learning model. The electronic medical record may include an electronic medical record graphical user interface that displays user health information, metabolic data, estimated event data, and actions. The user portal may include a user portal graphical user interface that allows the user 8 to view health information, receive notifications, and provide feedback. The mitigating action may include updating the electronic medical record graphical user interface to display an alert or notification to the healthcare provider 7 regarding a recommended intervention for the user 8. The mitigating action may cause the electronic medical record graphical user interface to display a recommended medication adjustment, a suggested dietary modification, or a physical activity recommendation based on the estimated event data. The user portal graphical user interface may be updated to display the mitigating action as a notification, reminder, or actionable item for the user 8. The mitigating action may trigger the user portal graphical user interface to present educational content related to the predicted past event, such as information about the effects of high carbohydrate foods on glucose levels or the timing of medication administration. The mitigating action may also cause the user portal graphical user interface to display a prompt for the user 8 to confirm or schedule a recommended action, such as ordering a medication refill from the pharmacy 9 or scheduling an appointment with the healthcare provider 7. The electronic medical record graphical user interface and the user portal graphical user interface may be rendered by rendered and/or executed by the electronic device 19 or the server 29.

The health management system 100 may use ambient listening through different devices to gather data without requiring the user 8 to enter data manually. Ambient listening may involve passive data collection from sensors, microphones, cameras, and other input devices associated with the electronic device 19. The ambient listening may capture audio data related to food consumption, physical activity, and environmental factors that influence metabolic data. In an example, the processor 301-1 may process ambient data captured by the electronic device 19 and transmit processed data to the server 29 via the network 32 for further analysis by the one or more models (e.g., a trained machine learning model, a generative machine learning model, etc.).

In some cases, the health management system 100 may interface with smart glasses that automatically take pictures of food when the user 8 is looking at food. The smart glasses may include a camera and image recognition capabilities to identify food items and estimate nutritional content. The smart glasses may communicate with the electronic device 19 or the server 29 via the network 32 to transmit food images and associated data. The one or more models (e.g., a trained machine learning model, a generative machine learning model, a classifying machine learning model, a classifier, etc.) may receive food data from the smart glasses as input for predicting metabolic data and generating actions.

The health management system 100 may use eye movement sensing from the smart glasses or some other electronic device 19 to correlate with stress or other mood indicators. Eye movement data may include gaze patterns, blink rates, pupil dilation, and saccade characteristics that may be indicative of stress, fatigue, or other mood states. The processor 301-1 or the processor 304-1 may process eye movement data to generate mood data that is input to the one or more models (e.g., a trained machine learning model, a generative machine learning model, a classifying machine learning model, a classifier, etc.) The mood data derived from eye movement sensing may be correlated with metabolic data to improve the accuracy of estimated event data.

The health management system 100 may use vibration sensing from the smart glasses or some other electronic device 19 to gather physiological data. Vibration sensing may detect subtle movements, tremors, or physiological signals that may be indicative of energy levels, stress states, or other health indicators. The vibration data may be transmitted to the server 29 via the network 32 and processed by the module 304-3 to generate energy data or mood data. The energy data and mood data derived from vibration sensing may be used as inputs to the one or more models (e.g., a trained machine learning model, a generative machine learning model, etc.) to improve predictions of past events and future metabolic data.

Referring to FIG. 3B, a training module 310 may be configured to train one or more machine learning models used by the health management system 100. The training module 310 may include training data 312, a training algorithm 320, and a training component 330. The training data 312 may comprise stage inputs 314 and known outcomes 318. The stage inputs 314 may be obtained from various sources including the healthcare provider 7, the servers 29, the electronic devices 19, the electronic medical record 14, outputs from processing steps, time in range values, time above range values, time below range values, severity scores, metabolic data classifications, glycemia risk index values, heart rate variability metrics, weight trend data, engagement data, etc.

The known outcomes 318 may be included for machine learning models generated based on supervised or semi-supervised training. An unsupervised machine learning model may not be trained using the known outcomes 318. The known outcomes 318 may include known or desired outputs for future inputs similar to or in the same category as the stage inputs 314 that do not have corresponding known outputs. The known outcomes 318 may include historical metabolic data, confirmed event data, and verified actions that resulted in mitigation of negative conditions.

With continued reference to FIG. 3B, the training data 312 and the training algorithm 320 may be provided to the training component 330. The training component 330 may apply the training data 312 to the training algorithm 320 to generate a machine learning model. The training component 330 may also receive comparison results 316 that compare a previous output of the corresponding machine learning model to apply the previous result to re-train the machine learning model. The comparison results 316 may be used by the training component 330 to update the corresponding machine learning model. The comparison results 316 may include differences between predicted metabolic data and actual metabolic data, differences between estimated event data and confirmed event data, differences between recommended actions and actions that resulted in successful mitigation of negative conditions, etc.

The training algorithm 320 may utilize machine learning networks and models including deep learning networks such as Deep Neural Networks, Convolutional Neural Networks, Fully Convolutional Networks, and Recurrent Neural Networks. The training algorithm 320 may also utilize probabilistic models such as Bayesian Networks and Graphical Models. The training algorithm 320 may further utilize discriminative models such as Decision Forests and maximum margin methods. The selection of the machine learning network or model may depend on the type of data being processed and the type of output being generated.

In some cases, the one or more models (e.g., a trained machine learning model, a generative machine learning model, etc.) may use a variational autoencoders architecture. The variational autoencoders architecture may utilize a framework of probabilistic graphical models to maximize a lower bound on the log likelihood of the historical metabolic data. The variational autoencoders architecture may encode input metabolic data into a latent representation and decode the latent representation to generate predicted metabolic data or estimated event data.

In some cases, the one or more models (e.g., a trained machine learning model, a generative machine learning model, etc.) may use an autoregressive models architecture. The autoregressive models architecture may specify that the output future metabolic data depends linearly on historical metabolic data and on a stochastic term. The stochastic term may represent an imperfectly predictable component of the metabolic data. The autoregressive models architecture may apply a stochastic difference or recurrence relationship to generate predictions of future metabolic data based on sequences of past metabolic data.

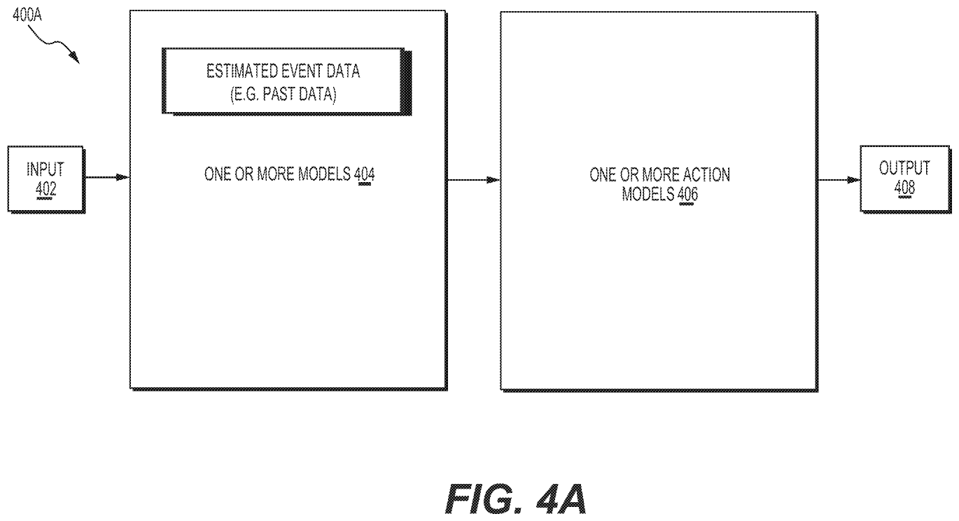

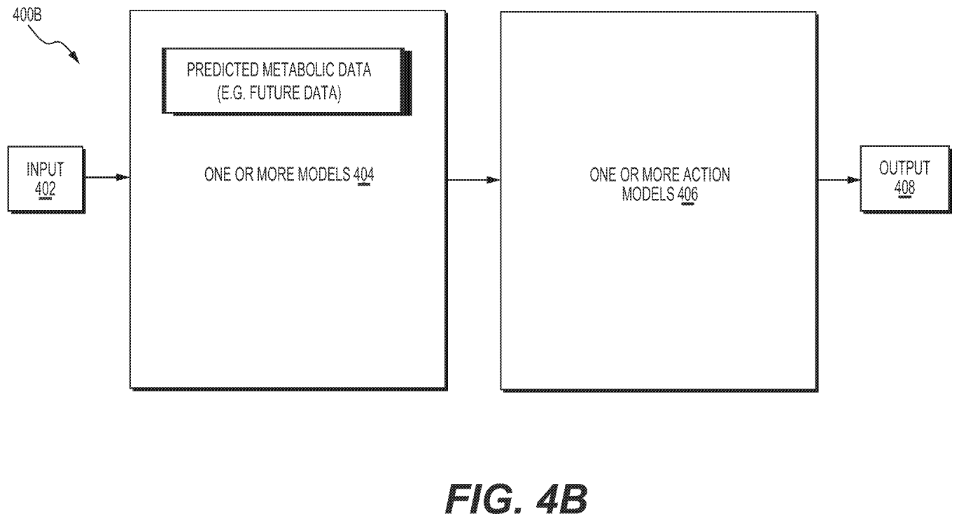

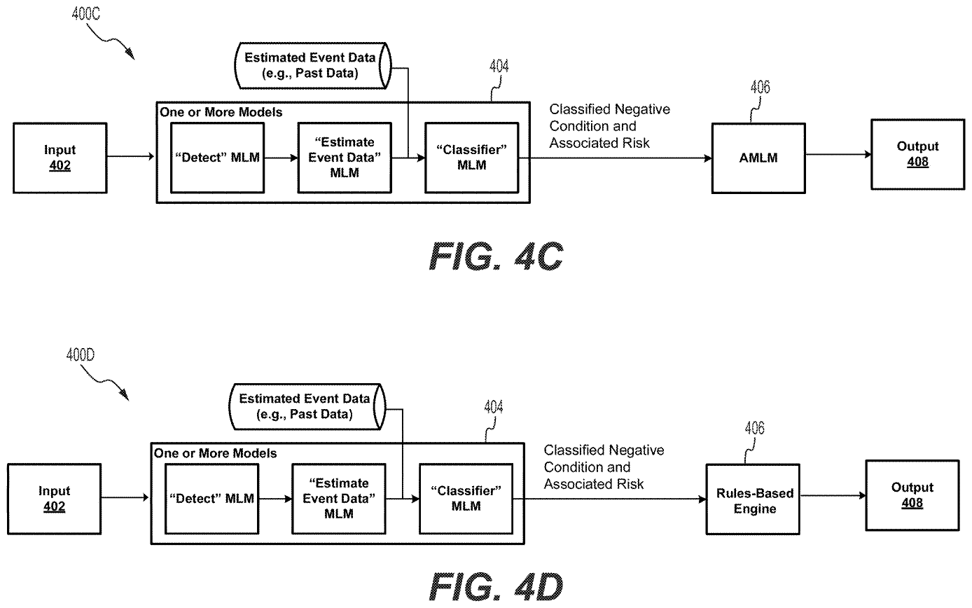

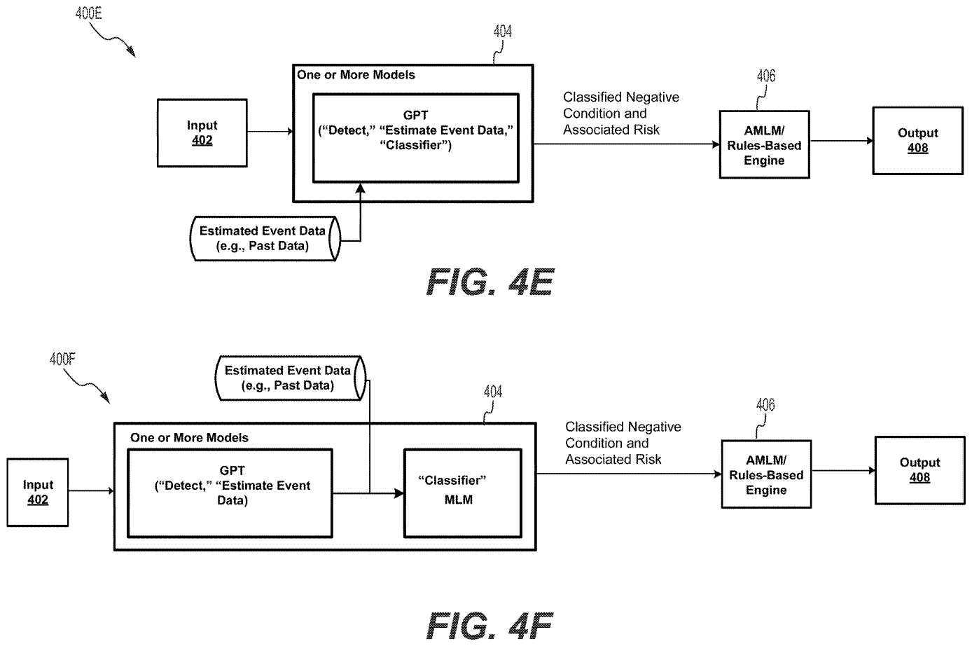

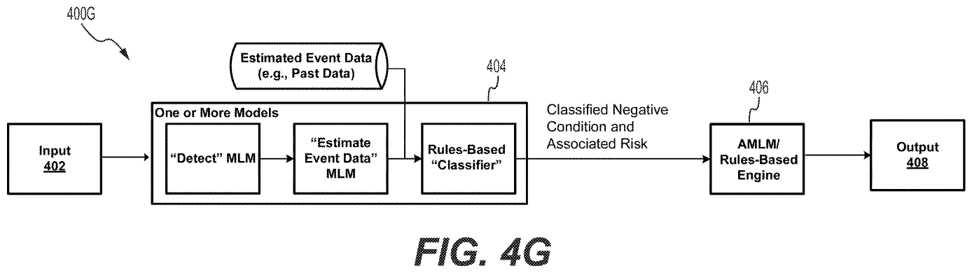

The health management system 100 may use one or more large language models (e.g., the one or more models discussed in reference to FIGS. 4A-4G) that are built using natural language processing techniques and trained on large quantities of unlabeled text using self-supervised or semi-supervised learning. The large language model may be designed to recognize and understand the structure and meaning of human language, classify texts according to content or purpose, and generate responses that are appropriate and coherent. The large language model may process textual representations of metabolic data, event data, and user information to generate predictions and actions.

In some cases, the health management system 100 may use clinical knowledge encoded and fine-tuned large language models including ClinicalBERT, Med-PaLM2, GatorTron, MedGPT, or Huatuo-GPT to train or supplement data provided to the machine learning models. The clinical knowledge encoded large language models may be trained on medical literature, clinical notes, and healthcare data to improve accuracy in processing metabolic data and generating clinically relevant actions.

In some cases, the action (e.g., mitigating action) output by the action machine learning model may be input into an optimization module. The optimization module may be a machine learning model, a feedback module, and/or part of a machine learning model that is configured to receive the mitigating action and additional mitigating actions associated with a plurality of users. The optimization module may be further configured to retrain the one or more models (e.g., a trained machine learning model, a generative machine learning model, a classifying machine learning model, a classifier, etc.)and the action machine learning model based on at least the mitigating action and the additional mitigating actions. The optimization module may aggregate mitigating actions from multiple users to identify patterns and improve the accuracy of estimated event data, predicted metabolic data, and actions generated by the one or more models (e.g., a trained machine learning model, a generative machine learning model, a classifying machine learning model, a classifier, etc.) and the action machine learning model. The retraining performed by the optimization module may occur periodically or in response to detection of model drift or decreased prediction accuracy.

A CGM device may include a sensor that is placed on or under the skin of a user to measure interstitial glucose levels. The sensor may measure glucose levels at regular intervals, such as every 1 to 5 minutes, and transmit the glucose data to a receiver or electronic device (e.g., electronic device 19). The CGM device may store glucose data over extended periods, such as 7 to 14 days, to provide a comprehensive view of glucose trends and patterns. The glucose data may be displayed on a graphical user interface that shows real-time glucose values, glucose trends, and historical glucose patterns. The CGM device may generate alerts when glucose levels exceed or fall below predetermined thresholds, such as hyperglycemic or hypoglycemic thresholds.

Similarly, a heart rate monitor may include a sensor that is worn on the body of a user to measure heart rate. The sensor may measure heart rate at regular intervals, such as every 1 to 5 seconds, and transmit the heart rate data to a receiver or electronic device (e.g., electronic device 19). The heart rate monitor may store heart rate data over extended periods, such as 7 to 30 days, to provide a comprehensive view of heart rate trends and patterns. The heart rate data may be displayed on a graphical user interface that shows real-time heart rate values, heart rate trends, and historical heart rate patterns. The heart rate monitor may generate alerts when heart rate exceeds or falls below predetermined thresholds, such as tachycardia or bradycardia thresholds. The heart rate monitor may also measure heart rate variability (HRV) data, which may provide insights into autonomic nervous system function, stress levels, and recovery status.

A smart scale may include a sensor platform that measures weight when a user stands on the scale. The smart scale may measure weight at regular intervals, such as daily or weekly, and transmit the weight data to a receiver or electronic device (e.g., electronic device 19). The smart scale may store weight data over extended periods, such as 30 to 90 days, to provide a comprehensive view of weight trends and patterns. The weight data may be displayed on a graphical user interface that shows current weight values, weight trends, and historical weight patterns. The smart scale may generate alerts when weight changes exceed predetermined thresholds, such as rapid weight gain or weight loss thresholds that may indicate fluid retention, dietary changes, or other health conditions. The smart scale may also measure additional metrics such as body composition, body mass index (BMI), or hydration levels.

In some cases, the CGM device may calculate metrics based on the glucose data, such as time in range, time above range, time below range, glucose variability, and average glucose. Time in range may represent the percentage of time that glucose levels remain within a target range, such as 70 to 180 mg/dL. Time above range may represent the percentage of time that glucose levels exceed the upper threshold of the target range. Time below range may represent the percentage of time that glucose levels fall below the lower threshold of the target range. Glucose variability may be calculated using metrics such as coefficient of variation or standard deviation of glucose values. Similarly, heart rate monitors may calculate metrics such as resting heart rate, average heart rate, heart rate variability, and time in target heart rate zones. Smart scales may calculate metrics such as average weight, weight trend, and weight variability. These metrics may be used as inputs to machine learning models to predict future metabolic data values and generate actions.

The CGM device may interface with other devices and systems to provide integrated health management. The CGM device may communicate with insulin pumps (via, for example, the network 32) to form a closed-loop insulin delivery system that automatically adjusts insulin delivery based on glucose levels. The CGM device may also communicate with mobile health applications (e.g., mobile health application 1), electronic medical records (e.g., electronic medical record 14), and cloud-based services to share glucose data and receive recommendations. The glucose data from the CGM device may be combined with other health data, such as heart rate, heart rate variability, weight, physical activity, sleep data, and dietary information, to provide a comprehensive view of the user's health status. Similarly, heart rate monitors and smart scales may interface with mobile health applications and cloud-based services to share metabolic data and receive recommendations.

Referring to FIG. 4A, a machine learning model system 400A may be configured for metabolic data based predictions. The machine learning model system 400A may include an input 402, one or more models 404 (e.g., a trained machine learning model, a generative machine learning model, a classifying machine learning model, a classifier, etc.), one or more action models 406 and an output 408. According to embodiments disclosed herein, the one or more models 404 may be or include a generative machine learning model, a trained machine learning model, a classifying machine learning model, a classifier (e.g., a rules based engine), etc. (generally referred to herein as a classifying component) and the one or more action models 406 may be or include an action machine learning model The machine learning model system 400A may form a system for detecting metabolic data, processing the metabolic data through cascading machine learning models, and outputting actions to mitigate negative conditions. In the configuration shown in FIG. 4A, the one or more models 404 may be trained to output estimated event data based on the metabolic data received as input 402.

The input 402 may include, for example, metabolic data for a user for a first time period. The metabolic data may include glucose levels measured via a CGM device over the first time period, heart rate data measured via a wearable device, heart rate variability data, weight data measured via a smart scale, or other metabolic data. The first time period may include approximately 6 hours, approximately 24 hours, or other time periods that provide sufficient data for prediction. The first time period may include a present time such that the metabolic data include current metabolic data. The input 402 may also include metadata associated with the user, such as age, gender, disease type, and race. The input 402 may further include event data that has been logged by the user or detected by sensors, such as food intake data, medication administration data, and exercise data. The input 402 may additionally include mood data comprising user-reported emotional states, physiological indicators of mood such as heart rate variability, and behavioral patterns associated with emotional conditions. The input 402 may include energy data comprising at least one of physiological data indicative of an energy state of the user, biometric data associated with fatigue of the user, metabolic expenditure data, at least one physiological parameter comprising heart rate, heart rate variability, respiration rate, skin conductance, blood oxygen level, body temperature, or movement intensity of the user, user-reported energy level data, or the like or a combination of such information. The input 402 may further include sleep data such as sleep duration, sleep quality metrics, sleep stages, and sleep onset and wake times. The input 402 may include stress data derived from cortisol levels, heart rate variability, skin conductance, or user-reported stress levels. The input 402 may also include environmental data such as ambient temperature, humidity, altitude, and air quality that may influence metabolic responses. The input 402 may include social context data such as meal timing relative to social events, work schedules, and travel information. The input 402 may further include historical health data such as prior diagnoses, comorbidities, family medical history, and previous treatment responses.

With continued reference to FIG. 4A, the one or more models 404 may receive, or sense, the input 402 and may be trained to output estimated event data based on the input 402. The estimated event data may include food data, exercise data, medication data, energy data, or mood data associated with the user. The food data may include a category of food consumed, such as high carbohydrate, medium carbohydrate, or low carbohydrate food. The food data may also include timing of food consumption and amount of food consumed. The exercise data may include type of exercise, duration of exercise, and intensity of exercise. The medication data may include type of medication, dosage of medication, and timing of medication administration. The energy data may include physiological data indicative of an energy state of the user, biometric data associated with fatigue of the user, and metabolic expenditure data. The mood data may include physiological data indicative of an emotional state of the user, biometric data correlated with mood of the user, heart rate variability data, skin conductance data, speech data, facial expression data, user-reported mood data, or combinations of such data.

The one or more models 404 may generate a probabilistic classification of likely causal events for certain metabolic data changes. The probabilistic classification may assign probabilities to different categories of events that may have caused observed changes in metabolic data. For example, the one or more models 404 may determine that there is a 70 percent probability that, based on the input 402, a glucose spike was caused by consumption of high carbohydrate food and a 20 percent probability that the glucose spike was caused by stress. As another example, the one or more models 404 may determine that there is an 80 percent probability that an elevated heart rate was caused by physical activity and a 15 percent probability that the elevated heart rate was caused by caffeine consumption. As yet another example, the one or more models 404 may determine that there is a 75 percent probability that a gradual weight increase over several days was caused by increased sodium intake and a 20 percent probability that the weight increase was caused by reduced physical activity. The probabilistic classification may be used to identify the most likely events that occurred during the first time period based on the pattern of metabolic data.

The one or more models 404 may use a sequence classification to categorize the input 402 for classifying events associated with a user. The sequence classification may analyze the temporal pattern of metabolic data to identify characteristic signatures associated with different types of events. For example, a rapid postprandial glucose rise followed by a gradual decline may be classified as indicative of high carbohydrate food consumption. As another example, a sudden increase in heart rate followed by a gradual return to baseline may be classified as indicative of physical exertion. As yet another example, a gradual increase in weight over multiple days may be classified as indicative of water retention or dietary changes. The sequence classification may also identify patterns associated with medication administration, exercise, sleep, and other events that influence metabolic data.

In some cases, the one or more models 404 may use MEDAL factors to infer estimated event data without requiring direct user input. MEDAL factors may include medication, exercise, diet, activity, and lifestyle factors that influence metabolic data. The one or more models 404 may be trained on historical data that includes both metabolic data and corresponding MEDAL factors to learn the relationships between metabolic patterns and underlying events. The one or more models 404 may then apply the learned relationships to infer MEDAL factors from new metabolic data without requiring the user to manually log events.

The one or more models 404 may be trained to classify a negative condition based on the detected anomalous event, the estimated event data, the metabolic data, and known event data associated with the user. The classification may include identifying the type of negative condition and determining a risk-stratification of the negative condition. The risk-stratification may classify the negative condition as low risk, moderate risk, high risk, or critical risk.

The risk-stratification may be based on factors such as the type and severity of the detected anomalous event, the estimated event data, the user's medical history, comorbidities, and current metabolic state. The risk-stratification may dictate the type of model used by the one or more action models 406 (e.g., an action machine learning model) in the second phase. For lower-risk applications, such as weight management recommendations, a machine learning model may be used to generate mitigating actions. For higher-risk applications, such as glucose management for insulin dosing, a rules-based engine may be used to generate mitigating actions until machine learning models demonstrate repeatable and sufficient accuracy for safety-critical applications.

The one or more models 404 (e.g., a classifying component) may output the classification and risk-stratification of the negative condition to the one or more action models 406. The one or more action models 406 may receive the classification and risk-stratification and may generate a mitigating action based on at least the classification and risk-stratification. The mitigating action may be further based on the detected event, the known and/or estimated events of interest during the first time period, and/or the predicted metabolic data.

The one or more models 404 may be implemented using various approaches depending on the risk level of the application and the type of negative condition being classified. In some cases, the one or more models 404 may be implemented as a rules-based engine that applies threshold-based logic to determine whether metabolic data indicates a negative condition. In some cases, the one or more models 404 may be implemented as a machine learning model that learns to classify negative conditions based on patterns in the metabolic data and estimated event data. In some cases, the one or more models 404 may be implemented as a large language model (LLM) that serves as a judge to evaluate whether metabolic data indicates a negative condition. The selection of the implementation approach may depend on factors such as the risk level of the application, regulatory requirements, and the need for adaptability to changing clinical guidelines.

For high-risk applications, such as glucose management or blood pressure management, the one or more models 404 (e.g., the classifying component) may be implemented as a rules-based engine. The rules-based engine may apply threshold-based logic to classify negative conditions based on predetermined clinical thresholds. For example, the rules-based engine may classify a negative condition when glucose values exceed a threshold such as approximately 250 mg/dL or when blood pressure values exceed clinically significant thresholds. The rules-based engine may provide deterministic and repeatable classification results, which may be advantageous for safety-critical applications where consistent behavior is required. For lower-risk applications, such as weight management, the one or more models 404 may be implemented as a machine learning model that learns to classify negative conditions based on patterns in the metabolic data. The machine learning model may provide more nuanced classification that accounts for individual variability and complex patterns in the metabolic data.

In some cases, when the one or more models 404 outputs predicted metabolic data for future time periods, the system may implement successive negative detection to reduce false positive alerts. When predicting future metabolic data values at regular intervals, such as approximately every 5 minutes, the system may avoid alerting the user if a predicted negative condition is expected to resolve shortly. The one or more models 404 may be configured to detect successive negative predictions before passing the classification to the one or more action models 406 (e.g., an action machine learning model, a rules-based engine, etc.). For example, when a first predicted metabolic data value indicates a negative condition, the system may record this as a first successive negative detection (N=1). When a second predicted metabolic data value at a subsequent interval also indicates a negative condition, the system may record this as a second successive negative detection (N=2). The system may continue to track successive negative detections until a threshold number of successive negatives is reached, such as three successive negative detections (N=3), before classifying the negative condition and passing the classification to the one or more action models 406. This successive negative detection approach may reduce unnecessary alerts when predicted negative conditions are transient and expected to resolve without intervention.

In some cases, the one or more models 404 (e.g., a classifying component) may be implemented as a large language model (LLM) that serves as a judge to evaluate whether metabolic data indicates a negative condition. The LLM-as-judge approach may provide flexibility in adapting to changing clinical guidelines or regulatory thresholds without requiring manual updates to a rules-based engine. For example, if clinical guidelines or regulatory thresholds change, such as a change in the glucose threshold from 250 mg/dL to 270 mg/dL, the LLM may adapt to the updated thresholds based on updated prompts or context information without requiring modifications to the underlying classification logic. The LLM-as-judge may receive the metabolic data, the estimated event data, and current clinical guidelines as inputs and may output a classification indicating whether the metabolic data indicates a negative condition. The LLM-as-judge may also output a risk-stratification indicating the severity of the negative condition. The classification and risk-stratification from the LLM-as-judge may then be provided to one or more action models 406 for generation of mitigating actions.

The one or more models 404 may use a transformer-based model to accurately predict metabolic data at various time intervals. The time intervals may include 0 to 5 minutes, 30 minutes, 60 minutes, 2 hours, 8 hours, or 24 hours. The transformer-based model may use attention mechanisms to contextualize the meaning of each metabolic data value in a sequence and predict subsequent metabolic data values. For example, the transformer-based model may predict future glucose values based on past glucose values, or may predict future heart rate values based on past heart rate values. The transformer-based model may be trained on large quantities of metabolic data from multiple users to learn general patterns of metabolic dynamics.

The one or more models 404 may also use a transformer-based model to determine the impact of events on metabolic data prediction at various time intervals. The transformer-based model may receive both metabolic data and event data as inputs 402 and may be trained to predict future metabolic data values based on the combined inputs. The transformer-based model may learn the relationships between different types of events and the resulting metabolic responses, enabling more accurate predictions when event data is available. For example, the transformer-based model may learn how different types of food affect glucose predictions, how different types of exercise affect heart rate predictions, or how medication timing affects weight predictions.

As further shown in FIG. 4A, the one or more action models 406 may receive the classification and risk-stratification of the negative condition from the one or more models 404. The one or more action models 406 (e.g., an action machine learning model) may be trained to output an action (e.g., mitigating action, etc.) to mitigate the negative condition based on at least the classification and risk-stratification. The negative condition may include hyperglycemia, hypoglycemia, glucose variability, elevated heart rate, abnormal heart rate variability, undesired weight change, mood disturbance, fatigue, or other adverse health states. The mitigating action may include an action property and an action time. The action property may specify the type of action to be performed, such as medication administration, food consumption, exercise, or notification. The action time may specify when the action should be performed to achieve the desired mitigation effect.

The machine learning model system 400A may employ a cascading architecture where a first model estimates event data, a second model classifies a negative condition and determines a risk-stratification, and a third model outputs a mitigating action based on the classification and risk-stratification. The one or more models 404 may function as the first model that estimates event data from metabolic data and as the second model that classifies the negative condition and determines the risk-stratification based on the estimated event data, the metabolic data, and known event data. The one or more action models 406 may function as the third model that generates actions based on the classification and risk-stratification. The cascading nature of the models may allow for detection of past events that caused prior metabolic changes, classification and risk-stratification of negative conditions, and generation of appropriate responses to mitigate the negative conditions.

Referring briefly to FIG. 4B, another machine learning model system 400B may be configured such that the one or more models 404 outputs predicted metabolic data instead of estimated event data. In this configuration, the one or more models 404 may receive the input 402, as previously discussed, including metabolic data (e.g., past metabolic data) and may be trained to output predicted metabolic data for a future time period. The predicted metabolic data may include predicted glucose values, predicted heart rate values, predicted heart rate variability values, predicted weight values, or other predicted physiological measurements. The one or more models 404 may classify a negative condition and determine a risk-stratification based on the predicted metabolic data. The one or more action models 406 may receive the classification and risk-stratification from the one or more models 404 and may be trained to output a mitigating action to mitigate the negative condition based on at least the classification and risk-stratification.

Referring to FIG. 4C, another machine learning model system 400C may be configured for metabolic data based predictions. The machine learning model system 400C may include one or more models 404, 406. The one or more models 404 may include a detection machine learning model, an estimate event data machine learning model, and a classifier machine learning model. The detection machine learning model may be configured to detect an anomalous event from the metabolic data received as input 402. The detection machine learning model may provide an output to the estimate event data machine learning model. The estimate event data machine learning model may be configured to estimate event data associated with the user during the first time period based on the detected anomalous event and the metabolic data. The estimate event data machine learning model may provide an output to the classifier machine learning model. The classifier machine learning model may receive the estimated event data from the estimate event data machine learning model and known event data associated with the user. The classifier machine learning model may be configured to classify a negative condition and determine a risk-stratification based on the detected anomalous event, the estimated event data, the metabolic data, and the known event data. The one or more models 404 may output the classified negative condition and associated risk-stratification to the one or more action models 406, which may be an action machine learning model. The one or more action models 406 may be configured to generate a mitigating action based on the classified negative condition and the risk-stratification. The output 408 may be the mitigating action from the one or more action models 406.

Referring to FIG. 4D, another machine learning model system 400D may be configured for metabolic data based predictions. The machine learning model system 400D may include one or more models 404, 406. The one or more models 404 may include a detection machine learning model, an estimate event data machine learning model, and a classifier machine learning model, configured in the same manner as described with reference to FIG. 4C. The detection machine learning model may provide an output to the estimate event data machine learning model, which may provide an output to the classifier machine learning model. Known event data may also be provided to the classifier machine learning model. The one or more models 404 may output the classified negative condition and associated risk-stratification to the one or more action models 406. In the machine learning model system 400D, the one or more action models 406 may be a rules-based engine rather than an action machine learning model. The rules-based engine may apply threshold-based logic or predetermined rules to generate a mitigating action based on the classified negative condition and the risk-stratification. The rules-based engine may provide deterministic and repeatable mitigating actions, which may be advantageous for safety-critical applications where consistent behavior is required. The output 408 may be the mitigating action from the rules-based engine.

Referring to FIG. 4E, another machine learning model system 400E may be configured for metabolic data based predictions. The machine learning model system 400E may include one or more models 404, 406. The one or more models 404 may include a single GPT model that is capable of performing tasks related to the detection machine learning model, the estimate event data machine learning model, and the classifier machine learning model. The single GPT model may receive the metabolic data as input 402 and known event data associated with the user. The single GPT model may be configured to detect an anomalous event from the metabolic data, estimate event data associated with the user during the first time period, and classify a negative condition based on the detected anomalous event, the estimated event data, the metabolic data, and the known event data. The single GPT model may output the classified negative condition and associated risk-stratification to the one or more action models 406. The one or more action models 406 may be either an action machine learning model or a rules-based engine. When the one or more action models 406 is an action machine learning model, the action machine learning model may generate a mitigating action based on the classified negative condition and the risk-stratification. When the one or more action models 406 is a rules-based engine, the rules-based engine may apply threshold-based logic or predetermined rules to generate a mitigating action. The output 408 may be the mitigating action from the action machine learning model or the rules-based engine.

Referring to FIG. 4F, another machine learning model system 400F may be configured for metabolic data based predictions. The machine learning model system 400F may include one or more models 404, 406. The one or more models 404 may include a GPT model and a classifier machine learning model. The GPT model may be capable of performing tasks related to the detection machine learning model and the estimate event data machine learning model. The GPT model may receive the metabolic data as input 402 and may be configured to detect an anomalous event from the metabolic data and estimate event data associated with the user during the first time period. The GPT model may provide an output to the classifier machine learning model. The classifier machine learning model may receive the detected anomalous event and the estimated event data from the GPT model and known event data associated with the user. The classifier machine learning model may be configured to classify a negative condition and determine a risk-stratification based on the detected anomalous event, the estimated event data, the metabolic data, and the known event data. The one or more models 404 may output the classified negative condition and associated risk-stratification to the one or more action models 406. The one or more action models 406 may be either an action machine learning model or a rules-based engine. The output 408 may be the mitigating action from the action machine learning model or the rules-based engine.

Referring to FIG. 4G, another machine learning model system 400G may be configured for metabolic data based predictions. The machine learning model system 400G may include one or more models 404, 406. The one or more models 404 may include a detection machine learning model, an estimate event data machine learning model, and a rules-based classifier. The detection machine learning model may be configured to detect an anomalous event from the metabolic data received as input 402. The detection machine learning model may provide an output to the estimate event data machine learning model. The estimate event data machine learning model may be configured to estimate event data associated with the user during the first time period based on the detected anomalous event and the metabolic data. The estimate event data machine learning model may provide an output to the rules-based classifier. The rules-based classifier may receive the estimated event data from the estimate event data machine learning model and known event data associated with the user. The rules-based classifier may apply threshold-based logic or predetermined rules to classify a negative condition and determine a risk-stratification based on the detected anomalous event, the estimated event data, the metabolic data, and the known event data. The one or more models 404 may output the classified negative condition and associated risk-stratification to the one or more action models 406. The one or more action models 406 may be either an action machine learning model or a rules-based engine. The output 408 may be the mitigating action from the action machine learning model or the rules-based engine.

Referring to both FIGS. 4A and 4B, the output 408 may be the mitigating action from the one or more action models 406. The output 408 may be transmitted to a medical device, a mobile health application, a notification system, or other components of the health management system 100. The medical device may be configured to perform a function upon receipt of the mitigating action, such as administering a medication. The medical device may be a wearable medical device, such as an insulin pump or a continuous glucose monitoring device with integrated insulin delivery capabilities. The mitigating action may also be transmitted to the electronic device 19 for display to the user 8 or to the servers 29 for storage and further processing.

The output 408 may be the mitigating action from the one or more action models 406 and may include recommended food information specifying a type of food, an amount of food, and a timing for food consumption to counteract a predicted glucose excursion. The mitigating action may include recommended exercise information specifying a type of physical activity, a duration of the physical activity, and an intensity level to help regulate glucose levels. The mitigating action may include recommended medicine information specifying a medication type, a dosage amount, and an administration time to address a predicted metabolic imbalance. The output 408 may be transmitted to a medical device, a mobile health application, a notification system, or other components of the health management system 100. The medical device may be configured to perform a function upon receipt of the mitigating action, such as administering a medication or providing an alert. The medical device may be a wearable medical device, such as an insulin pump or a continuous glucose monitoring device with integrated insulin delivery capabilities. The mitigating action may also be transmitted to the electronic device 19 for display to the user 8 or to the servers 29 for storage and further processing.

The output 408 may include facilitating a food order through integration with food delivery services or pharmacy systems. The facilitated food order may automatically order appropriate foods or nutritional supplements based on the predicted needs of the user. The output 408 may include activating and configuring a web or mobile application to display relevant health information, recommended food information specifying a type of food, an amount of food, and a timing for food consumption to counteract a predicted glucose excursion. The mitigating action may include recommended exercise information specifying a type of physical activity, a duration of the physical activity, and an intensity level to help regulate glucose levels. The mitigating action may include recommended medicine information specifying a medication type, a dosage amount, and an administration time to address a predicted metabolic imbalance.

The output 408 may include coaching provided through the mobile health application 1. The coaching may include personalized guidance regarding dietary choices, physical activity recommendations, medication timing adjustments, stress management techniques, or sleep hygiene practices based on the estimated event data. The coaching may be delivered through text notifications, audio messages, or interactive conversational interfaces.

The output 408 may include changes to graphical user interfaces displayed on the electronic device 19. The graphical user interface changes may include displaying alerts, warnings, or notifications regarding predicted negative conditions. The graphical user interface changes may include modifying the appearance of glucose trend displays to highlight areas of concern or predicted excursions. The graphical user interface changes may include presenting actionable recommendations with confirmation buttons for the user 8 to acknowledge or schedule recommended actions.

The output 408 may include updating the electronic medical record 14 or a user portal to reflect the estimated event data, predicted metabolic data, and recommended interventions. The update may trigger notifications to the healthcare provider 7 regarding significant predicted events or recommended treatment adjustments. The output 408 may include scheduling appointments with the healthcare provider 7 or initiating telemedicine consultations.

The output 408 may include adjusting settings on connected devices such as CGM alert thresholds, insulin pump basal rates, or activity tracker goals based on the estimated event data. The mitigating action may include providing educational content related to the estimated event data, such as information about the glycemic impact of specific foods or the timing of medication relative to meals.

The output 408 may include a medication dosage amount or a medication dosage timing determined based on the mitigating action. The medication dosage amount may specify a quantity of medication to be administered to the user to address the predicted negative condition. The medication dosage timing may specify when the medication should be administered relative to the current time, a meal, or another event. The medication dosage amount and the medication dosage timing may be transmitted to a medical device configured to deliver the medication to the user. The medical device may receive the medication dosage amount or the medication dosage timing and automatically administer the specified dosage at the specified time. The medical device may be an insulin pump that adjusts bolus or basal insulin delivery based on the received dosage information. The medication dosage amount and timing may be calculated by the one or more action models 406 based on the estimated event data and the user's historical response to medications.

The one or more models 404 and the one or more action models 406 may determine target metabolic data for the user over multiple time periods and predicted metabolic data for the user for multiple time periods, for example, a second time period. The second time period may be a future time period relative to the first time period from which the past metabolic data was sensed. The target metabolic data may represent target glucose values, target heart rate values, target weight values, or other target ranges that are clinically appropriate for the user based on factors such as the user's disease type, age, treatment goals, and historical metabolic patterns. The predicted metabolic data may represent the metabolic data values that the one or more models 404 predict will occur during the second time period based on, for example, the estimated event data and current metabolic trends.

The one or more models 404 (e.g., a classifying component)) may identify differences between the target metabolic data and the predicted metabolic data. The differences may include deviations in metabolic data values, timing of metabolic excursions, duration of time outside target ranges, or variability metrics. For example, the differences may include deviations in glucose values from target ranges, deviations in heart rate from resting heart rate targets, or deviations in weight from target weight ranges. The identified differences may indicate that the user is likely to experience a negative condition during the second time period if no intervention occurs.

Based on the identified differences, the one or more action models 406 may perform an action such as a subsequent mitigating action to mitigate a subsequent negative condition, after performing an initial mitigating action to mitigate an initial condition. The subsequent mitigating action may be proactive in nature, designed to prevent the predicted metabolic data from deviating from the target metabolic data. The subsequent mitigating action may include adjusting medication timing, recommending preemptive food consumption, suggesting physical activity, or modifying device settings to maintain glucose levels within the target range during the second time period.

The machine learning model systems 400A and 400B may be a system for detecting metabolic data, utilizing the one or more models 404 to output estimated event data or predicted metabolic data, and outputting a mitigating action to mitigate a negative condition based on the output. The system may provide an improved technology for mitigating negative conditions based on metabolic data and event data without material human intervention. The system may continuously sense metabolic data, process the metabolic data through the cascading machine learning models, generate mitigating actions, and perform the mitigating actions to maintain metabolic data within target ranges.

In some cases, the one or more models 404 may be configured to correlate estimated event data and predicted metabolic data. When the one or more models 404 is configured to output estimated event data, the one or more models 404 or a separate correlation machine learning model may be configured to generate predicted metabolic data based on the estimated event data. For example, if the one or more models 404 estimate that the user consumed a high carbohydrate meal at a particular time, the one or more models 404 or the separate correlation machine learning model may predict future glucose values based on the estimated food consumption event, the user's historical postprandial glucose responses, and the current glucose trajectory. Similarly, if the one or more models 404 estimate that the user engaged in physical activity, the one or more models 404 or the separate correlation machine learning model may predict future heart rate values and glucose values based on the estimated exercise event and the user's historical metabolic responses to similar activities.

Alternatively, when the one or more models 404 are configured to output predicted metabolic data, the one or more models 404 or a separate inference machine learning model may be configured to generate estimated event data based on the predicted metabolic data. For example, if the one or more models 404 predict an elevated heart rate in the near future, the one or more models 404 or the separate inference machine learning model may infer that the user is likely to engage in physical activity or has recently consumed caffeine that will cause the predicted heart rate elevation. As another example, if the one or more models 404 predict a weight increase over the coming days, the one or more models 404 or the separate inference machine learning model may infer that the user has increased sodium intake or reduced physical activity that will cause the predicted weight change. The correlation between estimated event data and predicted metabolic data may enable the one or more action models 406 to generate more accurate and contextually appropriate mitigating actions. The bidirectional relationship between estimated event data and predicted metabolic data may allow the machine learning model systems 400A and 400B to provide comprehensive insights into both the causes and consequences of metabolic changes, enabling proactive interventions that address underlying behaviors rather than merely responding to metabolic fluctuations.

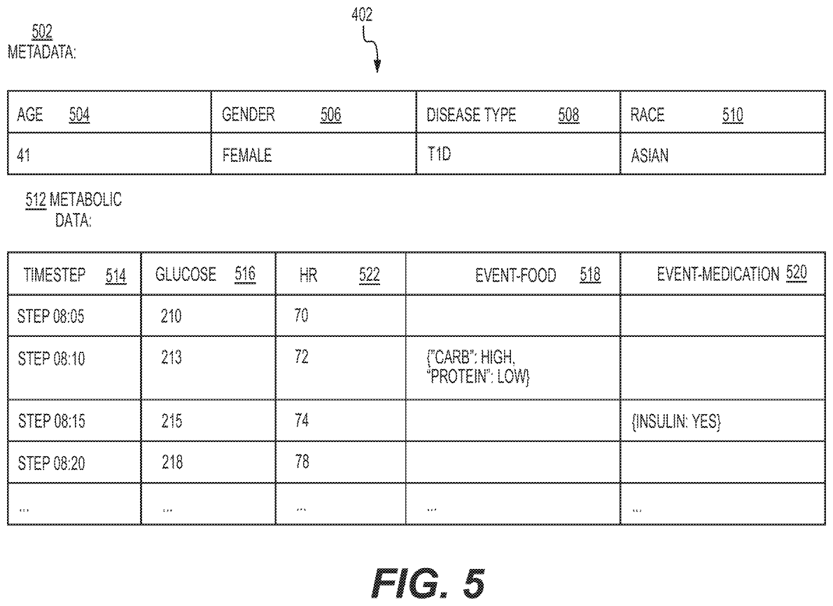

Referring to FIG. 5, the input 402 may include metadata 502, metabolic data 512, and other metabolic data related to a user. The metadata 502, the metabolic data 512, and other metabolic data may be organized in a structured format for input to the one or more models 404 (e.g., a generative machine learning model, a trained machine learning model, etc.) and the one or more action models 406 (e.g., an action machine learning model). The input 402 may represent data collected over a first time period, which may include approximately 6 hours or approximately 24 hours. The first time period may include a present time such that the input 402 includes current metabolic data in addition to historical metabolic data.

The metadata 502 may include user demographic and clinical information that provides context for interpreting the metabolic data 512. The metadata 502 may include an age field 504, a gender field 506, a disease type field 508, and a race field 510. The age field 504 may store a numerical value representing the user's age in years. As shown in FIG. 5, the age field 504 may contain a value such as 41. The gender field 506 may store a categorical value representing the user's gender, such as FEMALE or MALE. The disease type field 508 may store a categorical value representing the user's metabolic condition, such as T1D for Type 1 diabetes, T2D for Type 2 diabetes, or other disease classifications. The race field 510 may store a categorical value representing the user's racial or ethnic background, such as ASIAN, CAUCASIAN, AFRICAN AMERICAN, or other classifications.

The metabolic data 512 may include time-series data representing glucose measurements and associated events. The metabolic data 512 may include timesteps 514, glucose data 516, event-food data 518, event-medication data 520, heart rate data 522, and other metabolic-based data (e.g., weight). The timesteps 514 may include entries representing the time at which each metabolic data was recorded. The timesteps 514 may be formatted as time values, such as STEP 08:05, STEP 08:10, STEP 08:15, and STEP 08:20, representing measurements taken at 5-minute intervals. The glucose data 516 may contain numerical values representing the glucose concentration measured at each timestep 514, expressed in units such as mg/dL. The heart rate data 522 may contain numerical values representing the heart rate measured at each timestep 514, expressed in units such as beat per minute.

The event-food data 518 may include data representing food consumption events that occurred at or near the corresponding timestep 514. The event-food data 518 may be formatted as structured data indicating nutritional characteristics of consumed food. As shown in FIG. 5, the event-food data 518 may include an entry at STEP 08:10 indicating a meal high in carbohydrates and low in protein was consumed. The event-food data 518 may include additional nutritional information such as fat content, fiber content, glycemic index, portion size, or food category. No event-food data 518 may be recorded at timesteps at which no food consumption event was recorded.

The event-medication data 520 may include data representing medication administration events that occurred at or near the corresponding timestep 514. The event-medication data 520 may be formatted as structured data indicating the type of medication administered. As shown in FIG. 5, the event-medication data 520 may include an entry at STEP 08:15 indicating insulin administration. The event-medication data 520 may include additional medication information such as dosage amount, medication name, route of administration, or medication category. No event-medication data 520 may be recorded at timesteps at which no medication administration event was recorded.

The input 402 may include additional columns or fields beyond those shown in FIG. 5. The input 402 may include an event-exercise column containing data representing physical activity events, including exercise type, duration, and intensity. The input 402 may include an event-sleep column containing data representing sleep events, including sleep onset time, wake time, sleep duration, and sleep quality metrics. The input 402 may include an event-stress column containing data representing stress events, including stress level indicators derived from physiological measurements or user-reported data. The input 402 may include an event-mood column containing data representing mood states, including mood scores derived from standardized assessments such as PHQ-2 or PHQ-9 questionnaires. The input 402 may include an event-energy column containing data representing energy levels, including energy scores derived from user-reported data or physiological measurements.

The input 402 may include laboratory data such as HbA1c values, lipid panel results, kidney function markers, or other clinical measurements that provide context for interpreting glucose patterns. The input 402 may include device data such as CGM sensor calibration information, sensor age, or sensor accuracy metrics. The input 402 may include environmental data such as ambient temperature, humidity, or altitude that may influence glucose metabolism.

The metadata 502, the metabolic data 512, and other metabolic data may be used by the one or more models 404 to generate estimated event data or predicted metabolic data. The one or more models 404 may analyze patterns in the metabolic data 512 across the timesteps 514 to infer events that may have occurred but were not explicitly logged as, for example, event-food data 518 or event-medication data 520. The estimated event data may be stored, displayed, or presented to the user 8 for confirmation via a prompt enabling automated or semi-automated data logging and personalized feedback. The prompt may ask the user 8 to confirm whether a predicted food consumption event or medication administration event actually occurred, allowing the user 8 to correct or validate the estimated event data.

In some cases, the health management system 100 may generate a user-specific baseline metabolic profile for the user based on, for example, the metabolic data 512. The user-specific baseline metabolic profile may include characteristic fasting glucose patterns, postprandial glucose patterns, nocturnal glucose patterns, resting heart rate patterns, heart rate response patterns to activity, and weight fluctuation patterns. The fasting glucose patterns may represent typical glucose values and variability during periods without food consumption. The postprandial glucose patterns may represent typical glucose responses following meals, including peak glucose values, time to peak, and return to baseline. The nocturnal glucose patterns may represent typical glucose values and variability during sleep periods. The resting heart rate patterns may represent typical heart rate values during periods of rest. The weight fluctuation patterns may represent typical weight fluctuations from day-to-day based on, for example, a lever of hydration. The user-specific baseline metabolic profile may be generated for users to enable early detection of metabolic abnormalities.

The input 402 may include derived metrics calculated from the metabolic data 512 (glucose in this example). The derived metrics may include a time in range (TIR) value determined based on an amount of time a user's glucose level is within a threshold band over a base time period. The threshold band may be pre-determined based on clinical guidelines, such as 70 to 180 mg/dL for individuals with diabetes. The threshold band may be user-specific based on the user's treatment goals, disease type field 508, or historical glucose patterns. The threshold band may be dynamically determined based on factors such as time of day, recent food consumption, or physical activity level. The TIR value may be expressed as a percentage representing the proportion of time that glucose values remained within the threshold band during the base time period.

The input 402 may include a glycemia risk index (GRI) value determined based on a hypoglycemia component and a hyperglycemia component. The hypoglycemia component may be associated with an amount of time the user was hypoglycemic during a specified time period. Hypoglycemia may be defined as glucose values below a threshold such as 70 mg/dL or 54 mg/dL. The hyperglycemia component may be associated with an amount of time the user was hyperglycemic during the specified time period. Hyperglycemia may be defined as glucose values above a threshold such as 180 mg/dL or 250 mg/dL. The GRI value may combine the hypoglycemia component and the hyperglycemia component to provide a single metric representing overall glycemic risk. The GRI value may be weighted to reflect the relative clinical significance of hypoglycemia and hyperglycemia for the user based on factors such as disease type field 508, age field 504, or treatment goals.

The input 402 may include time above range values representing the percentage of time that glucose values exceeded the upper threshold of the target range. The input 402 may include time below range values representing the percentage of time that glucose values fell below the lower threshold of the target range. The input 402 may include glucose variability metrics such as coefficient of variation, standard deviation, or mean amplitude of glycemic excursions calculated from the glucose data 516 values. The derived metrics may be calculated by the electronic device 19, the server 29, or the CGM device and included in the input 402 for processing by the one or more models 404 and the one or more action models 406.

The input 402 may include derived metrics calculated from the heart rate data 522. The derived metrics may include a resting heart rate value determined based on heart rate measurements taken during periods of inactivity or sleep. The resting heart rate value may be calculated as an average of the lowest heart rate values recorded during a specified time period, such as during sleep or extended periods of rest. The input 402 may include heart rate variability (HRV) metrics calculated from the intervals between successive heartbeats. The HRV metrics may include time-domain measures such as standard deviation of normal-to-normal intervals (SDNN), root mean square of successive differences (RMSSD), or percentage of successive intervals differing by more than 50 milliseconds (pNN50). The HRV metrics may include frequency-domain measures such as low-frequency power, high-frequency power, or the ratio of low-frequency to high-frequency power. The HRV metrics may provide indicators of autonomic nervous system function, stress levels, and recovery status.

The input 402 may include heart rate zone data representing the amount of time the user spent in different heart rate intensity zones during a specified time period. The heart rate zones may be defined as percentages of maximum heart rate, such as a resting zone below 50 percent of maximum heart rate, a fat-burning zone between 50 and 70 percent of maximum heart rate, a cardio zone between 70 and 85 percent of maximum heart rate, and a peak zone above 85 percent of maximum heart rate. The maximum heart rate may be estimated based on the age field 504 or may be determined through fitness testing. The heart rate zone data may be used by the one or more models 404 to estimate exercise events or physical activity levels.

The input 402 may include derived metrics calculated from weight data. The derived metrics may include a weight trend value determined based on weight measurements taken over a specified time period, such as 7 days, 14 days, or 30 days. The weight trend value may indicate whether the user's weight is increasing, decreasing, or stable over the specified time period. The input 402 may include a weight variability metric representing the day-to-day fluctuation in weight measurements. The weight variability metric may be calculated as a standard deviation or coefficient of variation of daily weight measurements. High weight variability may indicate fluid retention, dietary inconsistencies, or measurement timing variations.

The input 402 may include body composition metrics derived from smart scale measurements. The body composition metrics may include body fat percentage, lean mass percentage, bone mass, or hydration level. The body composition metrics may provide additional context for interpreting weight changes and metabolic patterns. The input 402 may include a body mass index (BMI) value calculated from the user's weight and height. The BMI value may be used by the one or more models 404 and the one or more action models 406 to adjust predictions and recommendations based on the user's body composition category.

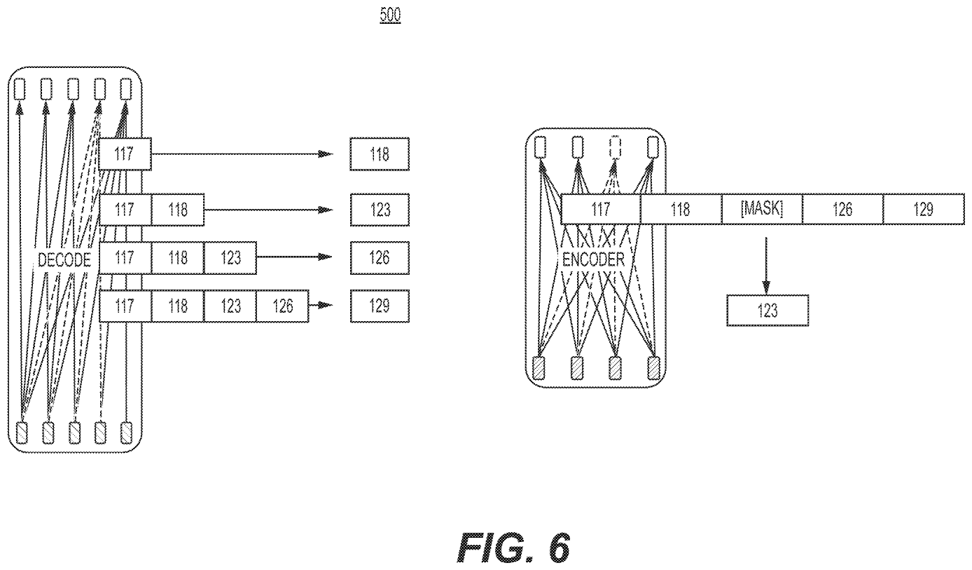

Referring to FIG. 6, a training system 600 may depict two different machine learning architectures for processing sequential data. The training system 600 may include a decoder-based architecture and an encoder-based architecture that may be used for training the one or more models 404 and the one or more action models 406 of the machine learning model systems 400A and 400B. The training system 600 may be implemented on the servers 29, the algorithm server 245, or other computing components of the health management system 100.

The decoder-based architecture may process input tokens in an autoregressive manner, where each subsequent row shows the progressive generation of output tokens. As shown in FIG. 6, the decoder-based architecture may process input tokens 117, 118, 123, 126, and 129 in sequence. The decoder-based architecture may receive input from below and generate outputs above, with arrows indicating the flow of information between layers. Dashed lines in the decoder-based architecture may represent attention connections between different positions in the sequence, showing how earlier tokens influence the prediction of later tokens. The decoder-based architecture may generate sequences one token at a time in a left-to-right fashion, where each generated token depends on all previously generated tokens. Here, the input tokens may include metabolic data such as glucose data 516 or heart rate data 522.

The decoder-based architecture may be used for next metabolic data prediction as a pre-train task. In next metabolic data prediction, the decoder-based architecture may receive a sequence of past metabolic data values and predict the next metabolic data value in the sequence. For example, the decoder-based architecture may receive a sequence of past glucose values and predict the next glucose value, or may receive a sequence of past heart rate values and predict the next heart rate value. The decoder-based architecture may learn to model the conditional probability distribution of each metabolic data value given all preceding metabolic data values. The autoregressive nature of the decoder-based architecture may allow the one or more models 404 to generate predictions for multiple future time steps by iteratively predicting one metabolic data value at a time and using each predicted value as input for predicting subsequent values.

The encoder-based architecture may process a sequence containing metabolic data tokens, such as glucose values 117, 118, a [MASK] token, 126, and 129, or heart rate values with similar structure. The encoder-based architecture may process all input tokens simultaneously, as indicated by bidirectional attention connections shown with dashed lines connecting all positions. The [MASK] token may represent a masked position that the encoder-based architecture is trained to predict, with an arrow pointing downward to token 123, indicating that the encoder-based architecture learns to predict the masked token based on the surrounding context. The encoder-based architecture may process entire sequences bidirectionally to predict masked elements based on full contextual information from both preceding and following tokens.

The encoder-based architecture may be used for masked metabolic data prediction as a pretrain task. In masked metabolic data prediction, one or more metabolic data values in a sequence may be replaced with a [MASK] token, and the encoder-based architecture may be trained to predict the original metabolic data values based on the surrounding context. The masked metabolic data prediction task may allow the one or more models 404 to learn bidirectional representations of metabolic data sequences, capturing relationships between metabolic data values that occur both before and after a given time point. The encoder-based architecture may learn to reconstruct missing metabolic data values based on patterns in the surrounding metabolic data, which may improve the ability of the one or more models 404 to infer estimated event data from incomplete or sparse metabolic data.

The training system 600 may demonstrate two fundamental approaches to sequence modeling that may be applied to metabolic data processing. The decoder-based architecture may be suited for tasks that involve generating future predictions based on past observations, such as predicting future glucose trajectories, future heart rate trajectories, or future weight trajectories. The encoder-based architecture may be suited for tasks that involve understanding the full context of a sequence, such as detecting events that occurred within a time period based on the complete metabolic data trace. The one or more models 404 may incorporate one or both of the decoder-based architecture and the encoder-based architecture depending on the specific task being performed.

The training system 600 may support multiple downstream tasks after pretraining with masked metabolic data prediction and next metabolic data prediction. A first downstream task may include metabolic data to metabolic data prediction, where given 24 hour metabolic data, the training system 600 may predict next 2 hour or 8 hour metabolic data values. For example, given 24 hour CGM data, the training system 600 may predict next 2 hour or 8 hour CGM data. Similarly, given 24 hour heart rate data, the training system 600 may predict next 2 hour or 8 hour heart rate values. The metabolic data to metabolic data prediction task may use the decoder-based architecture to generate future metabolic trajectories based on the learned representations from pretraining. The metabolic data to metabolic data prediction task may enable the health management system 100 to forecast metabolic data levels and identify potential negative conditions before the negative conditions occur.

A second downstream task may include metabolic data to event detection, where given 24 hour metabolic data, the training system 600 may detect when and what event occurred. The metabolic data to event detection task may use the encoder-based architecture to analyze the full metabolic data trace and classify events such as food intake, medication administration, exercise, or sleep based on characteristic patterns in the metabolic data. For example, given 24 hour CGM data, the training system 600 may detect food intake events based on postprandial glucose patterns. Given 24 hour heart rate data, the training system 600 may detect exercise events based on elevated heart rate patterns. The metabolic data to event detection task may correspond to the generation of estimated event data by the one or more models 404.

A third downstream task may include metabolic data to labels detection, where the training system 600 may detect disease type or user demographics based on metabolic data. The metabolic data to labels detection task may use the encoder-based architecture to classify users based on characteristic metabolic patterns associated with different disease types such as Type 1 diabetes or Type 2 diabetes. The metabolic data to labels detection task may also infer user demographics such as age or metabolic health status based on metabolic dynamics including glucose dynamics, heart rate dynamics, or weight dynamics.

The one or more models 404 may be trained based on historical metabolic data values for a cohort of individuals having Type 1 diabetes and may predict future metabolic data values for an individual having Type 2 diabetes. The training system 600 may use transfer learning to apply knowledge learned from one user population to another user population. The decoder-based architecture and the encoder-based architecture may learn general patterns of metabolic dynamics during pretraining that may be applicable across different disease types. The one or more models 404 may be fine-tuned on data from the target population to adapt the learned representations to the specific characteristics of the target disease type.

The one or more models 404 may be trained using supplementary variables including TIR values, GRI values, heart rate variability metrics, and weight trend metrics in addition to metabolic inputs. The supplementary variables may be incorporated into the input representations processed by the decoder-based architecture and the encoder-based architecture. The TIR values, GRI values, heart rate variability metrics, and weight trend metrics may provide summary metrics that capture overall metabolic control and risk, which may improve the ability of the one or more models 404 to predict future metabolic data values and detect events. The supplementary variables may be concatenated with metabolic data values at each timestep or may be provided as global context information that conditions the predictions of the one or more models 404.

The one or more models 404 may receive a supplementary condition as an input including an anticipated dosage and time for taking a given medication, an anticipated exercise, or an anticipated food to be consumed. The supplementary condition may be encoded and provided to the decoder-based architecture to condition the generation of future metabolic data predictions. The supplementary condition may allow the one or more models 404 to predict how metabolic data levels such as glucose levels, heart rate, or weight may respond to planned events, enabling the user 8 or the healthcare provider 7 to evaluate the expected impact of different actions before the actions are taken.

In an example, the training system 600 may implement a large sensor model (LSM)-GPT model that uses glucose data from both Type 1 diabetes and Type 2 diabetes populations in the training set to predict future glucose trajectories. LSMs may be foundation models trained on large quantities of sensor data to learn general representations of physiological signals and temporal patterns. LSMs may function by processing sequential sensor inputs through transformer-based architectures that capture long-range dependencies and contextual relationships within the data. In the context of the health management system 100, the LSM may be pretrained on diverse metabolic data streams including glucose data, heart rate data, and weight data from multiple users to learn universal patterns of metabolic dynamics. The LSM-GPT model may use the decoder-based architecture with a large sensor model trained on glucose data as the primary input. The LSM-GPT model may be trained using held-in and held-out training methodologies to evaluate prediction accuracy at various time intervals including 30 minutes, 60 minutes, and 2 hours. The LSM-GPT model may provide baseline glucose predictions based solely on glucose data without additional covariate information.

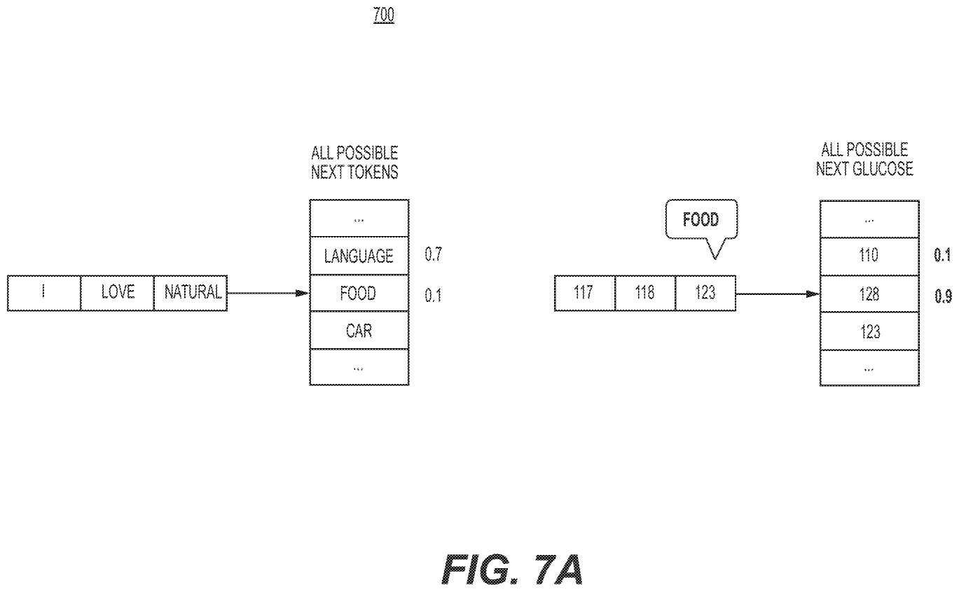

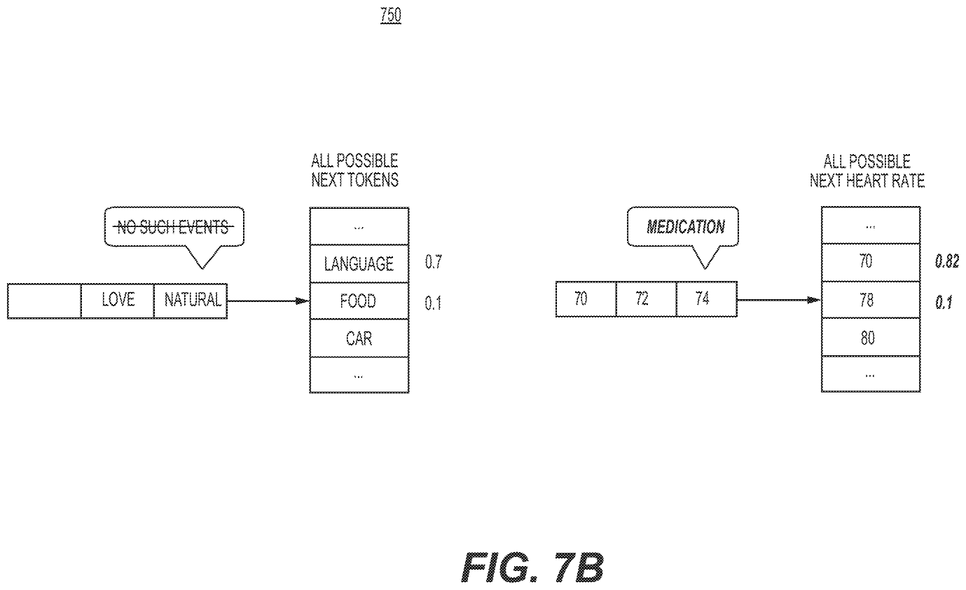

Referring to FIG. 7A, a first prediction example 700 may illustrate a system for determining an event and predicting future data related to the event. The first prediction example 700 may demonstrate a comparison between token prediction in a language model context and metabolic data value prediction in a metabolic data monitoring context. The first prediction example 700 may show how the one or more models 404 applies probabilistic prediction techniques to predict future metabolic data values based on prior metabolic data and event data.

On a left side of the first prediction example 700, a sequence of tokens including “I”, “LOVE”, and “NATURAL” may be shown as input, with an arrow pointing to a list of all possible next tokens. The possible next tokens may include “LANGUAGE” with a probability of 0.7, “FOOD” with a probability of 0.1, and “CAR” among other options. Here, a language model may assign probabilities to potential subsequent tokens in a sequence based on the context provided by preceding tokens. The language model may learn statistical patterns from training data to determine which tokens are most likely to follow a given sequence.

On a right side of the first prediction example 700, a “FOOD” token may be shown along with a sequence of glucose data including 117, 118, and 123, with an arrow pointing to a list of all possible next glucose values. The possible next glucose data may include 110 with a probability of 0.1, 128 with a probability of 0.9, and 123 among other options. Here, the one or more models 404 may assign probabilities to potential subsequent metabolic data values based on the context provided by preceding metabolic data values and event data such as food intake.

The first prediction example 700 may illustrate similarities between token prediction in language models and metabolic data value prediction in the one or more models 404. Both approaches may use sequential data as input and may generate probabilistic outputs representing the likelihood of different subsequent values. Both approaches may learn patterns from training data to determine conditional probability distributions over possible outputs given the input sequence. Both approaches may use attention mechanisms or recurrent architectures to capture dependencies between elements in the sequence.

The first prediction example 700 may also illustrate differences between token prediction in language models and metabolic data value prediction in the one or more models 404. Token prediction in language models may operate on discrete categorical values representing words or subwords, while metabolic data value prediction may operate on continuous numerical values representing metabolic data concentrations or measurements. Token prediction in language models may rely primarily on linguistic context, while metabolic data value prediction may incorporate additional context from event data such as food intake, medication administration, exercise, mood data, and energy data. The vocabulary of possible outputs in language models may be fixed and finite, while the range of possible metabolic data values may be continuous and unbounded within physiological limits.

The first prediction example 700 may demonstrate how the one or more models 404 generate a probabilistic classification of likely causal events for metabolic data changes. When the “FOOD” token is provided as input along with the glucose sequence, the one or more models 404 may determine that there is a 0.9 probability that the next glucose value is 128, reflecting the expected glucose rise following food consumption. The probabilistic classification may assign higher probabilities to metabolic data values that are consistent with the physiological response to the detected or reported event. The probabilistic classification may be used to identify the most likely metabolic data trajectory based on the pattern of metabolic data and associated event data from the input 402.

Referring to FIG. 7B, a second prediction example 750 may illustrate how different event types affect metabolic data predictions generated by the one or more models 404. The second prediction example 750 may demonstrate how when the health management system 100 receives information about a medication event rather than a food event, the probability distribution for predicted metabolic data values shifts accordingly. In the second prediction example 750, the metabolic data may include heart rate values to illustrate that the one or more models 404 may predict various types of metabolic data.

On a left side of the second prediction example 750, a sequence classification scenario may be presented where the system processes input tokens. The input sequence may show tokens including “NO SUCH EVENTS” (shown crossed out), “LOVE”, and “NATURAL” feeding into a list of all possible next tokens. The possible next tokens may include “LANGUAGE” with a probability of 0.7, “FOOD” with a probability of 0.1, “CAR”, and additional tokens. The crossed out “NO SUCH EVENTS” indication may demonstrate how the one or more models 404 adjusts predictions when certain event types are excluded from consideration.

On a right side of the second prediction example 750, a heart rate prediction scenario may be illustrated. The input 402 may include a “MEDICATION” event indicator along with a sequence of heart rate data shown as 70, 72, and 74 beats per minute. These values may feed into a list of all possible next heart rate data. The possible next heart rate data may include 70 with a probability of 0.82, 78 with a probability of 0.1, 80, and additional values. The second prediction example 750 may demonstrate how the one or more models 404 predict future heart rate data when a medication event is known to have occurred.

The second prediction example 750 may illustrate how the one or more models 404 adjust predictions based on different types of event data from the input 402 including food data, exercise data, and medication data. When the input 402 indicates a medication event such as a beta-blocker administration rather than a food event, the probability distribution for predicted heart rate data may shift to reflect the expected heart rate-lowering effect of the medication. In the second prediction example 750, the one or more models 404 may determine there is an 82 percent probability that the next heart rate value is 70 beats per minute and a 10 percent probability that the next heart rate value is 78 beats per minute, reflecting the impact of medication on heart rate predictions. The second prediction example 750 demonstrates that the one or more models 404 may predict various types of metabolic data including glucose values, heart rate values, weight values, and other physiological measurements.

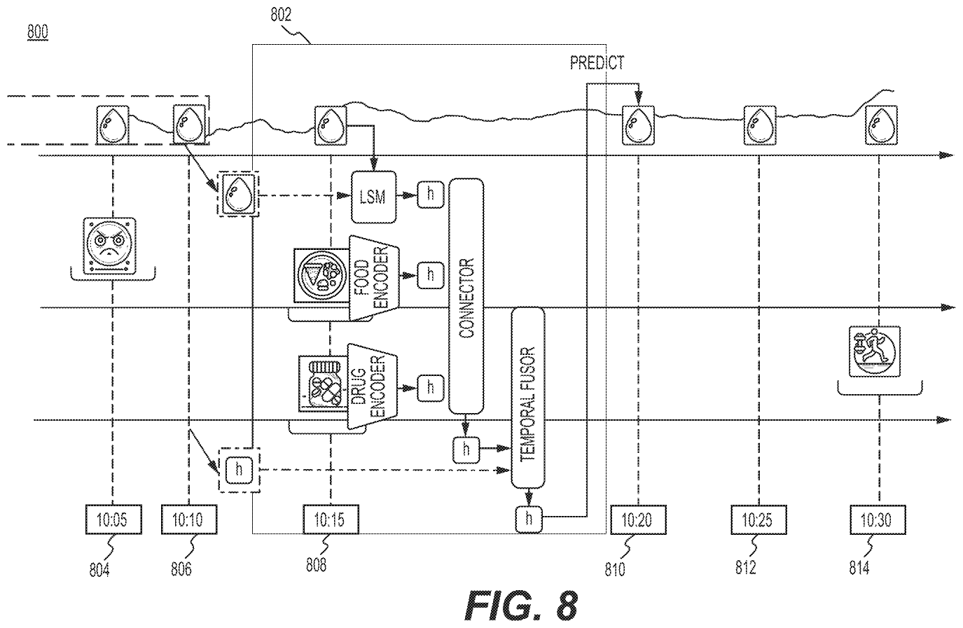

Referring to FIG. 8, a first user data example 800 may illustrate data flow and processing within a first metabolic data prediction system 802 (e.g., which may be or may be part of the one or more models 404). The first metabolic data prediction system 802 may be implemented as part of phase one discussed herein and may be configured to process metabolic data including glucose data, heart rate data, weight data, and other physiological measurements, and generate predictions based on multiple data inputs across multiple time periods. The first metabolic data prediction system 802 may correspond to or be implemented as the one or more models 404 of FIG. 4A-FIG. 4G. The components described below as being part of the first metabolic data prediction system 802 may include or interact with the one or more models 404.

The first metabolic data prediction system 802 may use a large metabolic data model architecture that includes multiple processing components for analyzing metabolic data and associated event data. The large metabolic data model architecture may integrate the functionality of the one or more models 404 into a unified prediction pipeline. The one or more models 404 may be implemented within the large metabolic data model architecture to process the metabolic data and generate estimated event data or predicted metabolic data. The large metabolic data model architecture may coordinate the data flow between these models to enable end-to-end processing from raw metabolic data inputs to actionable mitigating outputs.

The first metabolic data prediction system 802 may operate across multiple time periods including a first time period 804, a second time period 806, a third time period 808, a fourth time period 810, a fifth time period 812, and a sixth time period 814. The first time period 804 may correspond to a timestamp of 10:05, the second time period 806 may correspond to 10:10, the third time period 808 may correspond to 10:15, the fourth time period 810 may correspond to 10:20, the fifth time period 812 may correspond to 10:25, and the sixth time period 814 may correspond to 10:30. It will be understood that the time periods shown in FIG. 8 are different than the first time period discussed in reference to FIGS. 4A-4G (e.g., each of the time periods 804-814 may be part of the first time period referenced in FIGS. 4A-4G). Metabolic data readings, such as glucose readings or heart rate readings, may be captured at each of the time periods and represented by metabolic data value indicators positioned along a timeline. The time periods may represent 5-minute intervals at which metabolic data is sensed and processed by the first metabolic data prediction system 802.

The first metabolic data prediction system 802 may include several processing components arranged to receive and process different types of input data. The first metabolic data prediction system 802 may include a large sensor model (LSM) component that receives metabolic data as input and generates an output. The LSM component may process sequential metabolic data values from a CGM device, a heart rate monitor, a smart scale, or other monitoring devices to generate learned representations of metabolic dynamics. The LSM component may use transformer-based architectures or recurrent neural network architectures to capture temporal dependencies in the metabolic data.

The first metabolic data prediction system 802 may include a food encoder component that processes food-related event data and generates a corresponding output. The food encoder component may receive food data from the input 402 including food category, nutritional content, timing of food consumption, and portion size. The food encoder component may encode the food data into a representation that captures the expected glycemic impact of the consumed food. The food encoder component may learn relationships between different food types and corresponding glucose responses during training.

The first metabolic data prediction system 802 may include a drug encoder component that processes medication-related event data and generates a corresponding output. The drug encoder component may receive medication data from the input 402 including medication type, dosage amount, and timing of medication administration. The drug encoder component may encode the medication data into a representation that captures the expected effect of the medication on metabolic data levels. The drug encoder component may learn relationships between different medications, dosages, and corresponding metabolic responses during training.

The first metabolic data prediction system 802 may receive event data at specific time periods along the timeline. As shown in FIG. 8, a stress-related event may be indicated at the first time period 804. The stress-related event may represent a physiological or psychological stressor that influences metabolic data. The first metabolic data prediction system 802 may process the stress-related event data through the LSM component or through an additional encoder component configured to process stress data. The stress-related event may cause changes in metabolic data such as glucose levels or heart rate due to hormonal responses such as cortisol release, and the first metabolic data prediction system 802 may learn to predict metabolic data changes associated with stress events.

The multiple outputs from the LSM component, the food encoder component, the drug encoder component, and other encoder components may be directed to a connector component. The connector component may aggregate the outputs from the LSM component, the food encoder component, and the drug encoder component. The connector component may combine the learned representations from each encoder component into a unified representation that captures the combined effects of metabolic dynamics, food intake, and medication administration. The connector component may use concatenation, attention mechanisms, or other fusion techniques to integrate the outputs.

The connector component may forward the combined data to a temporal fusor component. The temporal fusor component may process the combined data from the connector component and generate predictions. The temporal fusor component may integrate temporal aspects of the combined data to account for the time-dependent effects of food and medication on metabolic data. The temporal fusor component may implement attention mechanisms that weight the relevance of different time points in the input sequence, enabling the first metabolic data prediction system 802 to identify which historical metabolic data values and events are most predictive of future metabolic data. The temporal fusor component may utilize recurrent neural network layers, long short-term memory (LSTM) cells, or transformer-based self-attention layers to capture long-range temporal dependencies between metabolic data values across the first time period 804, the second time period 806, the third time period 808, and subsequent time periods. The temporal fusor component may model how the effects of food and medication evolve over time and interact with underlying metabolic dynamics by learning pharmacokinetic and pharmacodynamic response curves from the training data 312. The temporal fusor component may receive the aggregated representations from the connector component and apply temporal convolution operations or gated recurrence operations to fuse information across multiple timesteps, thereby generating a temporally-aware representation that encodes both the current metabolic state and the anticipated trajectory of metabolic changes. The temporal fusor component may output this temporally-aware representation to a prediction head that generates predicted metabolic data for the fourth time period 810, the fifth time period 812, and the sixth time period 814. The first metabolic data prediction system 802 may include a prediction output that provides predicted metabolic data and/or estimated event data based on the processed inputs from the temporal fusor component, which may then be provided to the one or more action models 406 for generation of mitigating actions to address predicted negative conditions such as hyperglycemia, hypoglycemia, elevated heart rate, or abnormal weight change. The temporal fusor component may be responsible for fusing time-series information from multiple encoder outputs into a coherent temporal representation, while the one or more models 404 (e.g., a trained machine learning model, a generative machine learning model, etc.) may orchestrate the overall prediction pipeline including data preprocessing, encoding, temporal fusion, and output generation. In some implementations, the temporal fusor component may be one of several components that together constitute the one or more models 404, where the temporal fusor component performs the specific function of modeling temporal dependencies and the one or more models 404 performs the broader function of generating predictions or event estimates from raw metabolic data inputs.

The transformer-based large sensor model and the temporal fusor component described herein may represent specific technical implementations that are adapted to the characteristics of metabolic sensor data. Unlike general-purpose transformer architectures designed for natural language processing, the large sensor model may be specifically configured to process irregularly-sampled time-series data from physiological sensors, handle missing data points that may commonly occur in continuous glucose monitoring and heart rate monitoring, and capture the multi-scale temporal dependencies characteristic of metabolic processes. The temporal fusor component may implement attention mechanisms specifically designed to weight the relevance of different physiological events based on their temporal proximity and their known pharmacokinetic and pharmacodynamic relationships to metabolic outcomes. These technical adaptations may enable the machine learning models to achieve prediction accuracy that may not be possible using generic machine learning architectures applied to metabolic data.

The first metabolic data prediction system 802 may generate predicted metabolic data for each of the fourth time period 810, the fifth time period 812, and the sixth time period 814. The predicted metabolic data may indicate an anticipated metabolic trajectory based on the past metabolic data and detected event data processed through the LSM component, the food encoder component, the drug encoder component, and other encoder components. Based on the predicted metabolic data, the one or more action models 406 (e.g., an action machine learning model, a rules based engine, etc.) may determine that a mitigating action is appropriate to prevent or reduce a negative condition such as hyperglycemia, hypoglycemia, elevated heart rate, or abnormal weight change. The mitigating action may include an exercise-related event scheduled to be performed at the sixth time period 814. The one or more action models 406 may generate the exercise-related event as a recommended physical activity based on the predicted metabolic trajectory, where the exercise may cause changes in metabolic data such as a temporary spike in glucose followed by a decrease as muscles utilize glucose for energy, or an increase in heart rate followed by improved heart rate variability. The one or more action models 406 may determine the type, duration, and intensity of the recommended exercise based on the predicted metabolic data and the user's historical response to physical activity. The first metabolic data prediction system 802 may then incorporate the recommended exercise-related event into subsequent predictions to model the expected impact of the mitigating action on future metabolic data. This feedback loop between the one or more models 404 generating predicted metabolic data and the one or more action models 406 generating mitigating actions may enable proactive management of the user's metabolic state.

Predictions from the first metabolic data prediction system 802 may be used in conjunction with MEDAL characteristics where the user 8 may query the first metabolic data prediction system 802 requesting additional information about metabolic data and different activities or outcomes based on eating certain food or exercise. The user 8 may interact with the first metabolic data prediction system 802 through the mobile health application 1 or the conversational front end to ask questions about how different actions may affect metabolic data such as glucose levels, heart rate, or weight. The first metabolic data prediction system 802 may process the query and generate responses based on the learned relationships between MEDAL factors and metabolic dynamics.

The user 8 may query the first metabolic data prediction system 802 to see what MEDAL characteristics are available to maintain the current or predicted metabolic data value at a particular interval. The first metabolic data prediction system 802 may analyze the current metabolic state and predicted metabolic trajectory to identify actions that would maintain metabolic data within a target range. The first metabolic data prediction system 802 may recommend specific food types, exercise activities, or medication adjustments that would achieve the desired metabolic outcome. The recommendations may be generated by the one or more action models 406 based on the estimated event data and predicted metabolic data values from the first metabolic data prediction system 802.

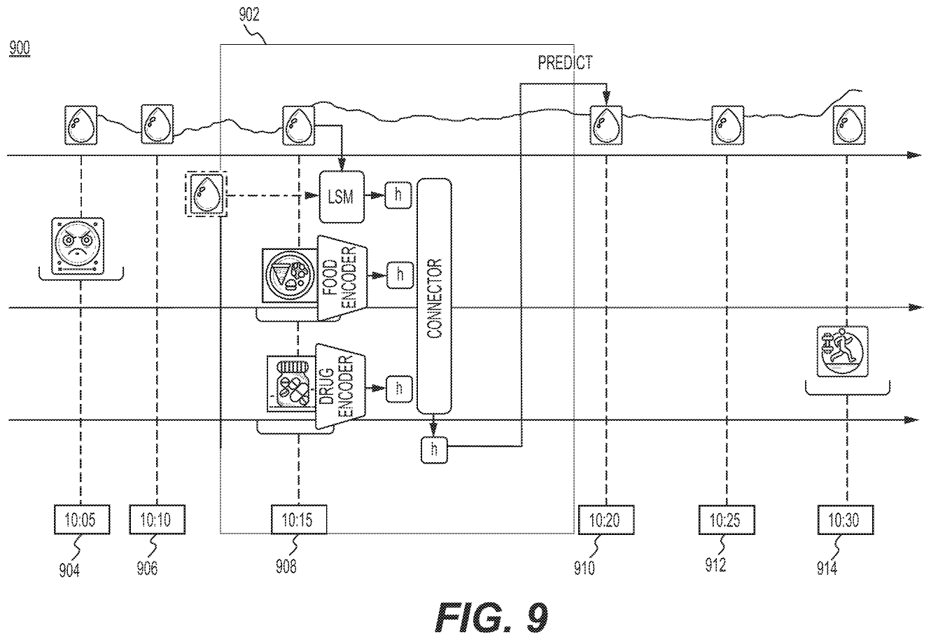

Referring to FIG. 9, a second user data example 900 may illustrate data flow and processing within a second metabolic data prediction system 902. The second metabolic data prediction system 902 may be implemented as part of phase one discussed herein and may be configured to process metabolic data including glucose data, heart rate data, weight data, and other physiological measurements, and generate predictions based on multiple data inputs across multiple time periods. The second metabolic data prediction system 902 may correspond to or be implemented as the one or more models 404 as described with reference to FIG. 4A-FIG. 4G. The second metabolic data prediction system 902 may use a small metabolic data model architecture that includes fewer processing components for analyzing metabolic data and associated event data than the first metabolic data prediction system 802 as described with reference to FIG. 8.

The second metabolic data prediction system 902 may operate across multiple time periods including a first time period 904, a second time period 906, a third time period 908, a fourth time period 910, a fifth time period 912, and a sixth time period 914. The first time period 904 may correspond to a timestamp of 10:05, the second time period 906 may correspond to 10:10, the third time period 908 may correspond to 10:15, the fourth time period 910 may correspond to 10:20, the fifth time period 912 may correspond to 10:25, and the sixth time period 914 may correspond to 10:30. It will be understood that the time periods shown in FIG. 9 are different than the first time period discussed in reference to FIGS. 4A-4G (e.g., each of the time periods 904-914 may be part of the first time period referenced in FIGS. 4A-4G).

The second metabolic data prediction system 902 may include processing components similar to those described with reference to the first metabolic data prediction system 802 of FIG. 8. The second metabolic data prediction system 902 may include the one or more models 404. The components described below as being part of the second metabolic data prediction system 902 may include or interact with the one or more models 404. The second metabolic data prediction system 902 may include a large sensor model (LSM) component, a food encoder component, a drug encoder component, and a connector component. The LSM component, the food encoder component, and the drug encoder component may function as described with reference to FIG. 8, where the LSM component processes metabolic data, the food encoder component processes food-related event data, and the drug encoder component processes medication-related event data.

The second metabolic data prediction system 902 may integrate with the one or more models 404 in a manner similar to the first metabolic data prediction system 802. The one or more models 404 may be implemented within the small metabolic data model architecture to process the metabolic data and generate estimated event data or predicted metabolic data. The small metabolic data model architecture may coordinate the data flow between these models to enable end-to-end processing from raw metabolic data inputs to actionable mitigating outputs, while utilizing fewer computational resources than the large metabolic data model architecture of the first metabolic data prediction system 802.

The second metabolic data prediction system 902 may differ from the first metabolic data prediction system 802 in that the second metabolic data prediction system 902 may use a small metabolic data model architecture that includes the LSM component, the food encoder component, the drug encoder component, and the connector component without a temporal fusor component. The small metabolic data model architecture may provide a more streamlined processing pipeline that combines outputs from the encoder components through the connector component and generates predictions directly without the additional temporal integration provided by the temporal fusor component. The small metabolic data model architecture may be suited for applications where computational resources are limited or where real-time prediction latency is a consideration.

Additionally or alternatively, the second metabolic data prediction system 902 may not receive stress-related event data as an input. The absence of stress-related event data may simplify the input processing for the second metabolic data prediction system 902 while still enabling predictions based on food, medication, and exercise factors.

The second metabolic data prediction system 902 may process the data from the LSM component, the food encoder component, and the drug encoder component to generate predicted metabolic trajectories at the fourth time period 910, the fifth time period 912, and the sixth time period 914. Based on the predicted metabolic data, the one or more action models 406 may determine that a mitigating action is appropriate to prevent or reduce a negative condition such as hyperglycemia, hypoglycemia, elevated heart rate, or abnormal weight change. The mitigating action may include an exercise-related event scheduled to be performed at the sixth time period 914.

Additionally or alternatively, based on the predicted metabolic trajectories, the second metabolic data prediction system 902 (via the one or more action models 406) may be used output, for example, suggestions on the types or amounts of the food ordered to maintain consistent or healthy metabolic data values. The suggestions may include recommendations to reduce portion sizes of high carbohydrate foods, substitute lower glycemic index alternatives, add protein or fiber to the order to moderate glucose response, or adjust the timing of food consumption relative to medication administration. The suggestions may also include recommendations to avoid caffeine or other stimulants to maintain healthy heart rate values, or recommendations regarding sodium intake to manage weight fluctuations. The suggestions may be displayed to the user 8 through the mobile health application 1 or through an interface integrated with a food delivery service.

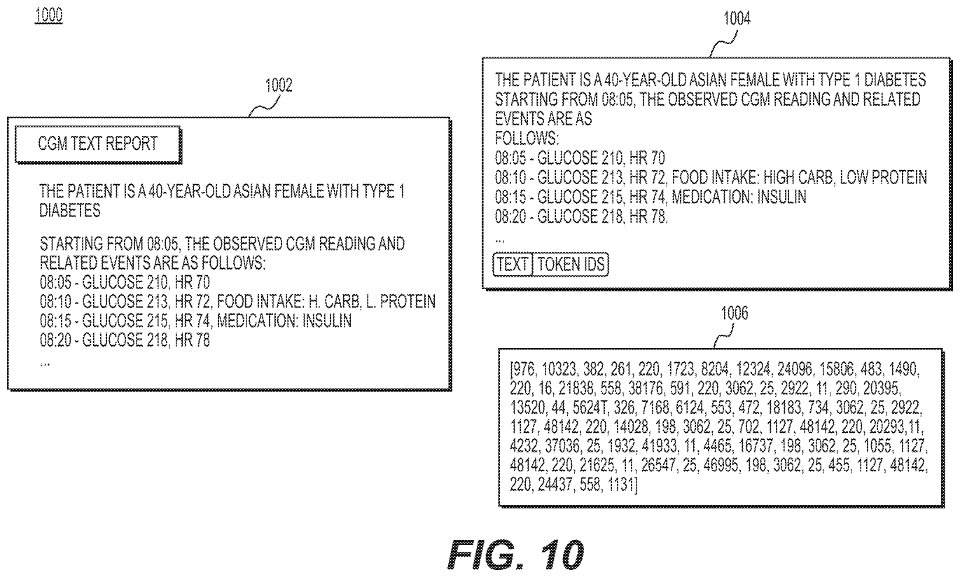

Referring to FIG. 10, data type examples 1000 may illustrate data type examples including a first data type 1002, a second data type 1004, and a third data type 1006. The data type examples 1000 may demonstrate how the health management system 100 generates a metabolic data text report containing user information, glucose readings, heart rate readings, and associated events or other readings, and how the health management system 100 converts textual data to numerical data for processing by the one or more models 404 and the one or more action models 406.

The first data type 1002 may include a metabolic data text report containing user demographic information and time-series metabolic data readings with associated event data. The metabolic data text report may indicate that the user is a 40-year-old Asian female with type 1 diabetes. The metabolic data text report may include timestamped glucose readings, heart rate readings, and related events starting from a specified time. The metabolic data text report may indicate that at 08:05 the glucose data is 210 and the heart rate is 70, at 08:10 the glucose data is 213 and the heart rate is 72 with a food intake event characterized as being high in carbohydrates and low in protein, at 08:15 the glucose data is 215 and the heart rate is 74 with a medication event indicating insulin administration, and at 08:20 the glucose data is 218 and the heart rate is 78. The metabolic data text report may continue with additional readings as indicated by ellipsis in the first data type 1002.

The first data type 1002 may represent a human-readable format of the metabolic data and event data that may be displayed to the user 8 or the healthcare provider 7 through the mobile health application 1 or the electronic medical record 14. The first data type 1002 may consolidate user metadata, metabolic data, and event data into a narrative format that provides context for interpreting the metabolic data readings. The first data type 1002 may be generated by the health management system 100 based on data stored in the database 302 or received from the CGM device, heart rate monitor, and the electronic device 19.

The second data type 1004 may present the same user information and metabolic data readings in a formatted text block. The second data type 1004 may display the user demographic data including age, gender, ethnicity, and disease type, followed by the timestamped glucose values, heart rate values, and associated events for food intake and medication. The second data type 1004 may include a label indicating text token identifiers, which may represent an intermediate representation between the human-readable text format and the numerical format processed by the machine learning models.

The third data type 1006 may display a numerical array representing text token identifiers corresponding to the textual data from the second data type 1004. The third data type 1006 may contain a sequence of numerical values enclosed in brackets. The numerical values may include values such as 976, 10323, 382, 261, 220, 1723, 8204, 12324, 24096, 15806, 483, 1490, and additional values as shown in FIG. 10. The text token identifiers may represent the encoded form of the textual CGM report data for processing by the one or more models 404 and the one or more action models 406.

The conversion from the first data type 1002 or the second data type 1004 to the third data type 1006 may represent a transformation of physiological properties sensed in a first format into metabolic data in a second format that may be utilized by the one or more models 404 and the one or more action models 406. The first format may include the human-readable textual representation of glucose readings, user demographics, and event data. The second format may include the numerical text token identifiers that encode the textual information into a format suitable for processing by transformer-based or other neural network architectures. The transformation may be performed by a tokenizer component that maps words, numbers, and symbols in the textual data to corresponding numerical identifiers from a vocabulary.

The text token identifiers in the third data type 1006 may encode semantic information from the textual metabolic data report. Each numerical value in the third data type 1006 may correspond to a word, subword, number, or symbol from the second data type 1004. The sequence of text token identifiers may preserve the order and structure of the original textual data, allowing, for example, the one or more models 404 to process the encoded representation and generate predictions based on the user information, glucose readings, heart rate readings, and event data contained in the metabolic data text report.

The first data type 1002, the second data type 1004, and the third data type 1006 may each be provided to the one or more models 404 and the one or more action models 406 as inputs. The one or more models 404 may process the third data type 1006 to generate estimated event data based on patterns in the encoded metabolic data readings and associated events. The one or more action models 406 may process outputs from the one or more models 404 to generate mitigating actions based on the estimated event data or predicted metabolic data.

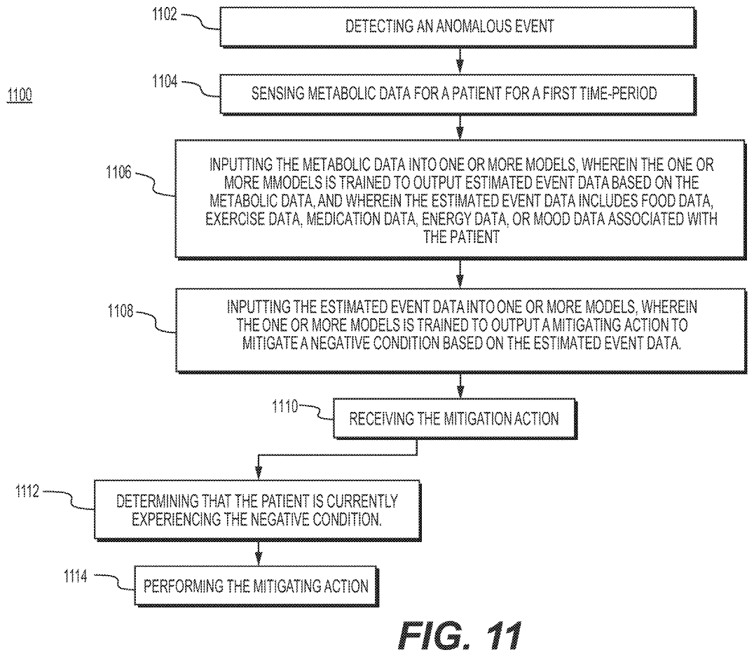

Referring to FIG. 11, a flowchart 1100 illustrates a method for analyzing metabolic-based events using estimated event data. The flowchart 1100 may correspond to the machine learning model system 400A configuration shown in FIG. 4A, where the one or more models 404 outputs estimated event data. The flowchart 1100 may include a first step 1102, a second step 1104, a third step 1106, a fourth step 1108, a fifth step 1110, a sixth step 1112, and a seventh step 1114. The flowchart 1100 may represent operations performed by the health management system 100, the machine learning model system 400A, the first metabolic data prediction system 802, the second metabolic data prediction system 902, or combinations thereof.

The first step 1102 may include detecting an anomalous event. The anomalous event may be detected by comparing the metabolic data to a user-specific baseline metabolic profile or to threshold values associated with negative conditions. The anomalous event may include a glucose spike, an elevated heart rate episode, an abnormal heart rate variability pattern, a rapid weight change, or other deviations from expected metabolic data patterns.

The detection of the anomalous event may be performed by one or more of various components of the health management system 100. In some cases, the one or more models 404 may perform the detection of the anomalous event as part of processing the metabolic data to generate estimated event data or predicted metabolic data. The one or more models 404 may identify characteristic patterns in the metabolic data that indicate anomalous events, such as glucose spikes, elevated heart rate episodes, or rapid weight changes. In some cases, a separate anomaly detection module or anomaly detection model may perform the detection of the anomalous event. The anomaly detection module may be configured to analyze the metabolic data and identify deviations from expected patterns before the metabolic data is provided to the one or more models 404. In some cases, the health management system 100 may perform the detection via comparison logic that compares the metabolic data to the user-specific baseline metabolic profile or to threshold values associated with negative conditions. The comparison logic may be implemented as rule-based logic, statistical analysis, or other algorithmic approaches that identify when metabolic data values exceed or fall below predetermined thresholds. In some cases, a combination of these components may perform the detection, where the health management system 100 applies threshold-based detection to identify potential anomalies, and the one or more models 404 or a separate anomaly detection model applies pattern recognition or machine learning-based detection to confirm or refine the detection. The detection may utilize various techniques including threshold-based detection, pattern recognition, statistical anomaly detection such as z-score analysis or interquartile range analysis, and machine learning-based detection approaches such as autoencoders, isolation forests, or recurrent neural networks trained to identify anomalous sequences in time-series metabolic data.

The second step 1104 may include sensing metabolic data for a user for a first time-period. The metabolic data may include glucose levels measured via a CGM device, heart rate data measured via a wearable device, heart rate variability data, weight data measured via a smart scale, or other metabolic data. The first time-period may include approximately 6 hours, approximately 24 hours, or other time periods that provide sufficient data for prediction.

The third step 1106 may include inputting the metabolic data into the one or more models 404. The one or more models 404 may be trained to output estimated event data based on the metabolic data. The estimated event data may include food data, exercise data, medication data, energy data, or mood data associated with the user. The energy data may comprise at least one of physiological data indicative of an energy state of the patient, biometric data associated with fatigue of the patient, metabolic expenditure data, at least one physiological parameter comprising heart rate, heart rate variability, respiration rate, skin conductance, blood oxygen level, body temperature, or movement intensity of the patient, or user-reported energy level data. The mood data may comprise at least one of physiological data indicative of an emotional state of the patient, biometric data correlated with mood of the patient, heart rate variability data, skin conductance data, speech data, facial expression data, or user-reported mood data.

The one or more models 404 may classify a negative condition based on the detected anomalous event, the estimated event data, the metabolic data, and known event data. The one or more models 404 may determine a risk-stratification of the negative condition as low risk, moderate risk, high risk, or critical risk. The classification and risk-stratification may be output to the one or more action models 406.

The fourth step 1108 may include inputting the classification and risk-stratification into the one or more action models 406 (e.g., an action machine learning model). The one or more action models 406 may receive the classification and risk-stratification from the one or more models 404 (e.g., a classifying component) The one or more action models 406 may be trained to output a mitigating action to mitigate the negative condition based on at least the classification and risk-stratification. The mitigating action may be further based on the estimated event data, the metabolic data, and/or known event data associated with the user. The negative condition may include hyperglycemia, hypoglycemia, glucose variability, elevated heart rate, abnormal heart rate variability, undesired weight change, mood disturbance, fatigue, tremor events, water weight retention, or other adverse health states. The mitigating action may include an action property and an action time. The action property may specify the type of action to be performed, such as medication administration, food consumption, exercise, notification, or device activation. The action time may specify when the action should be performed to achieve the desired mitigation effect.

The fifth step 1110 may include receiving the mitigating action from the one or more action models 406. The mitigating action may be received by the electronic device 19, the server 29, a medical device, or other components of the health management system 100. The mitigating action may include instructions for triggering one or more actions to address the negative condition.

The sixth step 1112 may include determining that the user is currently experiencing the negative condition. The determination may be based on current metabolic data sensed by the CGM device or other monitoring devices. The determination may be based on comparing current metabolic data to threshold values associated with the negative condition. The determination may be based on pattern recognition applied to recent metabolic data to identify signatures associated with the negative condition.

The seventh step 1114 may include performing the mitigating action. The mitigating action may be performed at an action time and in accordance with an action property. The mitigating action may be performed proactively based on the estimated event data or reactively in response to the determination that the patient is currently experiencing the negative condition.

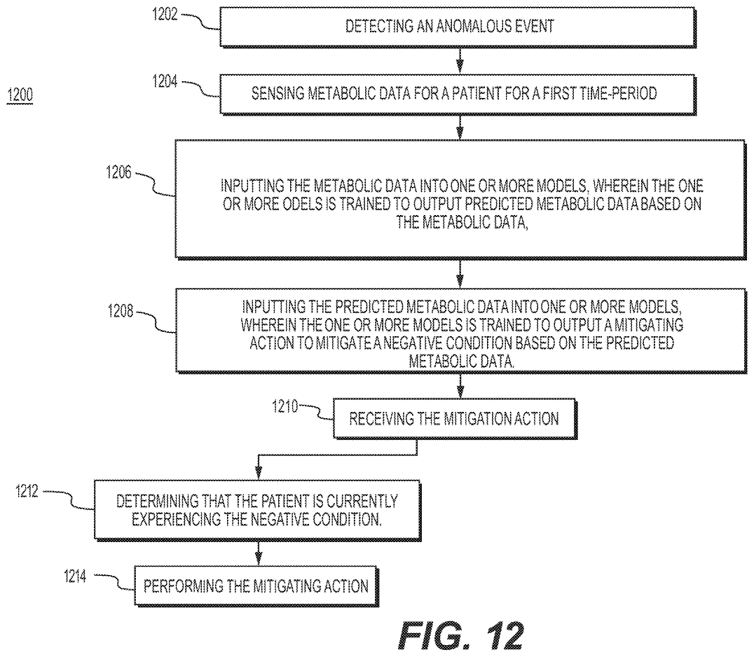

Referring to FIG. 12, a flowchart 1200 may illustrate a method for analyzing metabolic-based events using predicted metabolic data. The flowchart 1200 may correspond to the machine learning model system 400B configuration shown in FIG. 4B, where the one or more models 404 output predicted metabolic data. The flowchart 1200 may include a first step 1202, a second step 1204, a third step 1206, a fourth step 1208, a fifth step 1210, a sixth step 1212, and a seventh step 1214. The flowchart 1200 may represent operations performed by the health management system 100, the machine learning model system 400B, the first metabolic data prediction system 802, the second metabolic data prediction system 902, or combinations thereof.

The first step 1202 may include detecting an anomalous event. The anomalous event may be detected by comparing the metabolic data to a user-specific baseline metabolic profile or to threshold values associated with negative conditions. The anomalous event may include a glucose spike, an elevated heart rate episode, an abnormal heart rate variability pattern, a rapid weight change, or other deviations from expected metabolic data patterns.

The second step 1204 may include sensing metabolic data for a user for a first time-period. The metabolic data may include glucose levels measured via a CGM device, heart rate data measured via a wearable device, heart rate variability data, weight data measured via a smart scale, or other metabolic data. The first time-period may include approximately 6 hours, approximately 24 hours, or other time periods that provide sufficient data for prediction.

The third step 1206 may include inputting the metabolic data into the one or more models 404. The one or more models 404 may be trained to output predicted metabolic data based on the metabolic data. The predicted metabolic data may include predicted glucose values, predicted heart rate values, predicted heart rate variability values, predicted weight values, or other predicted physiological measurements for a future time period.

The one or more models 404 may classify a negative condition based on the detected anomalous event, the predicted metabolic data, the metabolic data, and known event data. The classifying machine learning model may determine a risk-stratification of the negative condition as low risk, moderate risk, high risk, or critical risk. The classification and risk-stratification may be output to the one or more action models 406.

The fourth step 1208 may include inputting the classification and risk-stratification into the one or more action models 406 (e.g., action machine learning model). The one or more action models 406 may receive the classification and risk-stratification from the one or more models 404. The one or more action models 406 (e.g., an action machine learning model) may be trained to output a mitigating action to mitigate the negative condition based on at least the classification and risk-stratification. The mitigating action may be further based on the predicted metabolic data, the metabolic data, and/or known event data associated with the user. The negative condition may include hyperglycemia, hypoglycemia, glucose variability, elevated heart rate, abnormal heart rate variability, undesired weight change, mood disturbance, fatigue, tremor events, water weight retention, or other adverse health states. The mitigating action may include an action property and an action time. The action property may specify the type of action to be performed, such as medication administration, food consumption, exercise, notification, or device activation. The action time may specify when the action should be performed to achieve the desired mitigation effect.

The fifth step 1210 may include receiving the mitigating action from one or more action models 406. The mitigating action may be received by the electronic device 19, the server 29, a medical device, or other components of the health management system 100. The mitigating action may include instructions for triggering one or more actions to address the negative condition.

The sixth step 1212 may include determining that the user is currently experiencing the negative condition. The determination may be based on current metabolic data sensed by the CGM device or other monitoring devices. The determination may be based on comparing current metabolic data to threshold values associated with the negative condition. The determination may be based on pattern recognition applied to recent metabolic data to identify signatures associated with the negative condition.

The seventh step 1214 may include performing the mitigating action. The mitigating action may be performed at an action time and in accordance with an action property. The mitigating action may be performed proactively based on the predicted metabolic data or reactively in response to the determination that the patient is currently experiencing the negative condition.

The mitigating action output by the one or more action models 406 may be transmitted to a medical device (e.g., the electronic device(s) 19) configured to perform a function upon receipt of the mitigating action. The medical device may receive the mitigating action through a wireless communication protocol such as Bluetooth, Wi-Fi, near-field communication, or cellular network connectivity (e.g., the network 32). The medical device may be configured to interpret the mitigating action and execute corresponding operations to address the predicted negative condition. The transmission of the mitigating action to the medical device may occur automatically without requiring manual intervention from the user or the healthcare provider 7.

The function performed by the medical device may include administering a medication using the medical device. The medical device may be an insulin pump configured to deliver insulin to the user based on the mitigating action. The medical device may be a medication infusion device configured to deliver other medications such as glucagon, hormones, or therapeutic agents. The medical device may receive instructions specifying the type of medication to administer, the dosage amount, and the timing of administration. The medical device may execute the medication administration function by activating pumping mechanisms, opening valves, or triggering injection systems to deliver the specified medication to the user.

The medical device may be a wearable medical device. The wearable medical device may be worn on the body of the user and may include sensors for monitoring physiological parameters and actuators for delivering therapeutic interventions. The wearable medical device may be an insulin pump worn on the abdomen, arm, or other body location. The wearable medical device may be a patch pump that adheres to the skin and delivers medication through a subcutaneous cannula. The wearable medical device may be an implanted medical device positioned beneath the skin or within body tissues to provide continuous medication delivery. The wearable medical device may communicate with the health management system 100 to receive mitigating actions and report delivery status and user response data.

A medication dosage amount or a medication dosage timing may be determined based on the mitigating action. The medication dosage amount may specify a quantity of medication to be administered to the user to address the predicted negative condition. The medication dosage amount may be calculated based on the estimated event data, the user's historical response to medications, and user-specific factors such as body weight, age, and disease type. The medication dosage timing may specify when the medication should be administered relative to the current time, a meal, or another event. The medication dosage timing may be calculated to optimize the therapeutic effect of the medication based on pharmacokinetic and pharmacodynamic models.

The medication dosage amount or the medication dosage timing may be transmitted to the medical device configured to deliver a medication to the user based on the medication dosage amount or the medication dosage timing. The medical device may receive the medication dosage amount and automatically prepare the specified quantity of medication for delivery. The medical device may receive the medication dosage timing and schedule the medication delivery to occur at the specified time. The medical device may adjust basal delivery rates, bolus amounts, or infusion schedules based on the received medication dosage amount and medication dosage timing. The medical device may provide confirmation to the health management system 100 that the medication has been delivered according to the specified parameters.

A medication may be determined based on the mitigating action, and an automated order may be triggered via an ordering system for the medication. The ordering system may be integrated with pharmacy systems, medication delivery services, or healthcare supply chains. The automated order may specify the medication type, quantity, dosage form, and delivery address based on the mitigating action and user information. The automated order may be transmitted to the pharmacy 9 for fulfillment and delivery to the user. The automated order may include refill requests for medications that the user is currently prescribed when the mitigating action indicates that medication supplies may be depleted. The ordering system may verify insurance coverage, check for drug interactions, and confirm prescription validity before processing the automated order.

The mitigating action may include adjusting a temperature in a location. The temperature adjustment may be transmitted to a thermostat, climate control system, or smart home device configured to regulate environmental temperature. The temperature adjustment may be determined based on predicted past event data indicating that environmental temperature may influence the user's metabolic state. The temperature adjustment may be implemented to create conditions that support glucose regulation, such as maintaining a cooler temperature to promote metabolic activity or a warmer temperature to reduce physiological stress. The temperature adjustment may be coordinated with other mitigating actions such as exercise recommendations or medication timing to optimize the overall therapeutic effect.

An automated communication may be triggered via a communication channel to at least one of a contact or an emergency service. The communication channel may include text messaging, phone calls, push notifications, email, or emergency alert systems. The automated communication may be directed to different recipients depending on the risk associated with the negative condition. For higher risk negative conditions, the automated communication may contact a hospital, emergency medical services, or on-call healthcare providers. For lower risk negative conditions, the automated communication may contact a family member, caregiver, or designated emergency contact. The automated communication may include information about the predicted negative condition, the recommended mitigating action, and instructions for the recipient regarding appropriate responses.

The electronic medical record 14 or a user portal may be updated based on the mitigating action. The update to the electronic medical record 14 may include recording the estimated event data, the predicted negative condition, the recommended mitigating action, and the outcome of the mitigating action. The update may create an audit trail documenting the automated interventions performed by the health management system 100. The update may trigger alerts or notifications within the electronic medical record system to inform healthcare providers of significant events or recommended treatment adjustments. The user portal may be updated to display the mitigating action as a notification, reminder, or actionable item for the user. The user portal may present educational content related to the estimated event data and the recommended mitigating action to help the user understand the rationale for the intervention.

A number of implementations have been described. Nevertheless, it will be understood that various modifications may be made without departing from the spirit and scope of the disclosure. Accordingly, other implementations are within the scope of the following claims.

In accordance with the above disclosure, the following embodiments are provided:

Embodiment 1. A method, comprising: detecting an anomalous event from metabolic data received from a user during a first time period; estimating event data associated with the user during the first time period, wherein the estimated event data includes food data, exercise data, medication data, energy data, or mood data associated with the user and contemporaneous with the metabolic data, wherein the estimated event data relates to unknown event data associated with the user during the first time period; classifying a negative condition based on the detected anomalous event, the metabolic data, the estimated event data, and known event data associated with the user during the first time period; and generating a mitigating action based on the negative condition, wherein the mitigating action mitigates the negative condition.

Embodiment 2. The method of embodiment 1, wherein the estimating event data, classifying a negative condition, and generating a mitigating action are performed by one of a same machine learning model or two or more different machine learning models.

Embodiment 3. The method of embodiment 1, wherein the mitigating action is transmitted to a medical device, the medical device configured to perform a function upon receipt of the mitigating action.

Embodiment 4. The method of embodiment 1, wherein the detecting the anomalous event, the estimating the event data, and the classifying the negative condition are performed by one or more models.

Embodiment 5. The method of embodiment 4, wherein the one or more models comprise a first model configured to detect the anomalous event, a second model configured to estimate the event data, and a third model configured to classify the negative condition.

Embodiment 6. The method of embodiment 4, wherein the one or more models comprise a model configured to detect the anomalous event, estimate the event data, and classify the negative condition.

Embodiment 7. The method according to embodiment 1, wherein the classifying comprises: identifying the negative condition; and determining a risk-stratification of the negative condition.

Embodiment 8. The method of embodiment 1, wherein the generating the mitigating action includes triggering an automated communication via a communication channel to at least one of a contact or an emergency service.

Embodiment 9. The method according to embodiment 1, wherein the detecting, estimating, and classifying are performed iteratively, and the generating the mitigating action is performed when a negative condition is classified during two or more consecutive classifications.

Embodiment 10. The method according to embodiment 1, further comprising: generating a user-specific baseline profile based on the metabolic data; comparing current metabolic data to the user-specific baseline profile to identify a deviation; and detecting the anomalous event based on the deviation.

Embodiment 11. The method according to embodiment 1, wherein estimating the event data is performed by a generative machine learning model, and wherein the generative machine learning model is trained based on historical metabolic data for a cohort of users having a health condition.

Embodiment 12. A non-transitory computer-readable medium having computer-executable instructions stored thereon, wherein the instructions include the steps comprising: detecting an anomalous event from metabolic data received from a user during a first time period; estimating event data associated with the user during the first time period, wherein the estimated event data includes food data, exercise data, medication data, energy data, or mood data associated with the user and contemporaneous with the metabolic data, wherein the estimated event data relates to unknown event data associated with the user during the first time period; classifying a negative condition based on the detected anomalous event, the metabolic data, the estimated event data, and known event data associated with the user during the first time period; and generating a mitigating action based on the negative condition.

Embodiment 13. The non-transitory computer-readable medium of embodiment 12, wherein the estimating event data, classifying a negative condition, and generating a mitigating action are performed by one of a same machine learning model or two or more different machine learning models.

Embodiment 14. The non-transitory computer-readable medium of embodiment 12, wherein the mitigating action is transmitted to a medical device, the medical device configured to perform a function upon receipt of the mitigating action.

Embodiment 15. The non-transitory computer-readable medium of embodiment 14, wherein the function includes administering a medication using the medical device.

Embodiment 16. The non-transitory computer-readable medium of embodiment 12, wherein a first model is configured to detect the anomalous event and estimate the event data and a second model is configured to classify the negative condition.

Embodiment 17. A system for generating a mitigating action, the system comprising: a trained machine learning model; a classifier; an action machine learning model; a memory having processor-readable instructions stored therein; and one or more processors configured to access the memory and execute the processor-readable instructions, which, when executed by the one or more processors, configures the one or more processors to perform a method, the method comprising: detecting an anomalous event from metabolic data received from a user during a first time period; estimating, by the trained machine learning model, event data associated with the user during the first time period, wherein the estimated event data includes food data, exercise data, medication data, energy data, or mood data associated with the user and contemporaneous with the metabolic data, wherein the estimated event data relates to unknown event data associated with the user during the first time period; classifying, by the classifier, a negative condition based on the detected anomalous event, the metabolic data, the estimated event data, and known event data associated with the user during the first time period; and generating, by the action machine learning model, a mitigating action based on the negative condition.

Embodiment 18. The system of embodiment 17, wherein the mitigating action is transmitted to a medical device, the medical device configured to perform a function upon receipt of the mitigating action.

Embodiment 19. The system of embodiment 17, wherein the detecting the anomalous event, the estimating the event data, and the classifying the negative condition are performed by one or more models.

Embodiment 20. The system of embodiment 17, wherein the classifying comprises: identifying the negative condition; and determining a risk-stratification of the negative condition.

Embodiment 21. A method, comprising: detecting an anomalous event from metabolic data received from a user during a first time period; estimating event data associated with the user during the first time period, wherein the estimated event data includes food data, exercise data, medication data, energy data, or mood data associated with the user and contemporaneous with the metabolic data, wherein the estimated event data relates to unknown event data associated with the user during the first time period; predicting metabolic data associated with the user during a second time period based on the detected anomalous event, the received metabolic data, the estimated event data, and known event data associated with the user during the first time period, wherein the predicted metabolic data includes at least one of glucose levels measured via a continuous glucose monitoring (CGM) device, heart rate data, heart rate variability (HRV) data, ketone data, weight data, cortisol data, hormone data, body electrical data, respiratory data, blood pressure data, body temperature data, or oxygen saturation data; classifying a negative condition based on the detected anomalous event, the metabolic data, the estimated event data, known event data associated with the user during the first time period, and the predicted metabolic data; and generating a mitigating action based on the classified negative condition to mitigate occurrence of the negative condition during the second time period.

Embodiment 22. The method of embodiment 21, further comprising, prior to classifying the negative condition, detecting that at least a first predicted metabolic data of the predicted metabolic data and a second predicted metabolic data of the predicted metabolic data indicate the negative condition.

Embodiment 23. The method of embodiment 21, wherein the detecting the anomalous event, the estimating the event data, the predicting the metabolic data, the classifying the negative condition, and the generating the negative condition are performed by one or more models.

Embodiment 24. The method of embodiment 21, wherein a first model is configured to detect the anomalous event, a second model is configured to estimate the event data and predict the metabolic data, a third model is configured to classify the negative condition, and a fourth model to generate the mitigating action.

Embodiment 25. A method, comprising: receiving metabolic data from a user during a first time period; predicting metabolic data associated with the user during a second time period based on the received metabolic data, the estimated event data, and known event data associated with the user during the first time period, wherein the predicted metabolic data includes at least one of glucose levels measured via a continuous glucose monitoring (CGM) device, heart rate data, heart rate variability (HRV) data, ketone data, weight data, cortisol data, hormone data, body electrical data, respiratory data, blood pressure data, body temperature data, or oxygen saturation data; classifying a negative condition based on the metabolic data, the estimated event data, known event data associated with the user during the first time period, and the predicted metabolic data; and generating a mitigating action based on the classified negative condition to mitigate occurrence of the negative condition during the second time period.

Embodiment 26. The method of embodiment 25, further comprising, prior to classifying the negative condition, detecting that at least a first predicted metabolic data of the predicted metabolic data and a second predicted metabolic data of the predicted metabolic data indicate the negative condition.

Embodiment 27. The method of embodiment 25, wherein the predicting the metabolic data, the classifying the negative condition, and the generating the mitigating action are performed by one or more models.

Embodiment 28. The method of embodiment 25, wherein a first model is configured to predict the metabolic data, a second model is configured to classify the negative condition, and a third model is configured to generate the mitigating action.

What is claimed is:

1. A method, comprising:
detecting an anomalous event from metabolic data received from a user during a first time period;
estimating event data associated with the user during the first time period, wherein the estimated event data includes food data, exercise data, medication data, energy data, or mood data associated with the user and contemporaneous with the metabolic data, wherein the estimated event data relates to unknown event data associated with the user during the first time period;
classifying a negative condition based on the detected anomalous event, the metabolic data, the estimated event data, and known event data associated with the user during the first time period; and
generating a mitigating action based on the negative condition, wherein the mitigating action mitigates the negative condition.

2. The method of claim 1, wherein the estimating event data, classifying a negative condition, and generating a mitigating action are performed by one of a same machine learning model or two or more different machine learning models.

3. The method of claim 1, wherein the mitigating action is transmitted to a medical device, the medical device configured to perform a function upon receipt of the mitigating action.

4. The method of claim 1, wherein the detecting the anomalous event, the estimating the event data, and the classifying the negative condition are performed by one or more models.

5. The method of claim 4, wherein the one or more models comprise a first model configured to detect the anomalous event, a second model configured to estimate the event data, and a third model configured to classify the negative condition.

6. The method of claim 4, wherein the one or more models comprise a model configured to detect the anomalous event, estimate the event data, and classify the negative condition.

7. The method according to claim 1, wherein the classifying comprises:
identifying the negative condition; and
determining a risk-stratification of the negative condition.

8. The method of claim 1, wherein the generating the mitigating action includes triggering an automated communication via a communication channel to at least one of a contact or an emergency service.

9. The method according to claim 1, wherein the detecting, estimating, and classifying are performed iteratively, and the generating the mitigating action is performed when a negative condition is classified during two or more consecutive classifications.

10. The method according to claim 1, further comprising:
generating a user-specific baseline profile based on the metabolic data;
comparing current metabolic data to the user-specific baseline profile to identify a deviation; and
detecting the anomalous event based on the deviation.

11. The method according to claim 1, wherein estimating the event data is performed by a generative machine learning model, and wherein the generative machine learning model is trained based on historical metabolic data for a cohort of users having a health condition.

12. A non-transitory computer-readable medium having computer-executable instructions stored thereon, wherein the instructions include the steps comprising:
detecting an anomalous event from metabolic data received from a user during a first time period;
estimating event data associated with the user during the first time period, wherein the estimated event data includes food data, exercise data, medication data, energy data, or mood data associated with the user and contemporaneous with the metabolic data, wherein the estimated event data relates to unknown event data associated with the user during the first time period;
classifying a negative condition based on the detected anomalous event, the metabolic data, the estimated event data, and known event data associated with the user during the first time period; and
generating a mitigating action based on the negative condition.

13. The non-transitory computer-readable medium of claim 12, wherein the estimating event data, classifying a negative condition, and generating a mitigating action are performed by one of a same machine learning model or two or more different machine learning models.

14. The non-transitory computer-readable medium of claim 12, wherein the mitigating action is transmitted to a medical device, the medical device configured to perform a function upon receipt of the mitigating action.

15. The non-transitory computer-readable medium of claim 14, wherein the function includes administering a medication using the medical device.

16. The non-transitory computer-readable medium of claim 12, wherein a first model is configured to detect the anomalous event and estimate the event data and a second model is configured to classify the negative condition.

17. A system for generating a mitigating action, the system comprising:
a trained machine learning model;
a classifier;
an action machine learning model;
a memory having processor-readable instructions stored therein; and
one or more processors configured to access the memory and execute the processor-readable instructions, which, when executed by the one or more processors, configures the one or more processors to perform a method, the method comprising:
detecting an anomalous event from metabolic data received from a user during a first time period;
estimating, by the trained machine learning model, event data associated with the user during the first time period, wherein the estimated event data includes food data, exercise data, medication data, energy data, or mood data associated with the user and contemporaneous with the metabolic data, wherein the estimated event data relates to unknown event data associated with the user during the first time period;
classifying, by the classifier, a negative condition based on the detected anomalous event, the metabolic data, the estimated event data, and known event data associated with the user during the first time period; and
generating, by the action machine learning model, a mitigating action based on the negative condition.

18. The system of claim 17, wherein the mitigating action is transmitted to a medical device, the medical device configured to perform a function upon receipt of the mitigating action.

19. The system of claim 17, wherein the detecting the anomalous event, the estimating the event data, and the classifying the negative condition are performed by one or more models.

20. The system of claim 17, wherein the classifying comprises:
identifying the negative condition; and
determining a risk-stratification of the negative condition.
